(19) United States
(12) Patent Application Publication
(10) Pub. No.: US 2020/0300805 A1
(43) Pub. Date: Sep. 24, 2020

(54) MICROELECTRONIC SENSOR FOR SENSING ELECTRICAL SIGNALS IN SUB-TERAHERTZ AND TERAHERTZ FREQUENCY RANGES

(71) Applicant: EPITRONIC HOLDINGS PTE. LTD., Singapore (SG)

(72) Inventors: Ayal RAM, Singapore (SG); Walid-Madhat MUNIEF, Blieskastel (DE)

(21) Appl. No.: 16/808,778

(22) Filed: Mar. 4, 2020

Related U.S. Application Data
(60) Provisional application No. 62/821,855, filed on Mar. 21, 2019.

(51) Int. Cl. G01N 27/414; G01N 33/68

(52) U.S. Cl. CPC G01N 27/4143; G01N 2033/0095; G01N 27/4145; G01N 33/6818

(57) ABSTRACT

The present invention describes a method for chemical sensing and biomolecular diagnostics with a microelectronic sensor based on the combination of an open-gate pseudo-conductive high-electron mobility transistor and a Vivaldi antenna installed in the open gate area of the transistor and operated in the sub-THz and THz frequency range.

### TECHNICAL FIELD

The present application relates to the field of microelectronic sensors based on high-electron-mobility transistors and their use in detection and continuous monitoring of electrical signals in sub-terahertz and terahertz frequency ranges for chemical sensing and biomolecular diagnostics. In particular, the present application relates to the microelectronic sensor combining an open-gate pseudo-conducting high-electron-mobility transistor (PC-HEMT) and Vivaldi antenna for chemical sensing and biomolecular diagnostics.

### BACKGROUND

### Chemical Sensing and Biomolecular Diagnostics

Chemical sensing is likely the most primordial sensory modality that emerged in the evolution of life. Without chemical sensing life on earth would probably not exist. It is used for detecting nutrients, avoiding threats, finding mating partners and various forms of communication and social interaction between animals.

The advent of artificial sensors has created a myriad of problems in the areas of chemical detection and identification with applications in food quality and pollution control, chemical threat detection, health monitoring, robot control and even odour and taste synthesis. Efficient algorithms are needed to address many challenges of chemical sensing in these areas, including (but not limited to) sensitivity levels, sensor drift, concentration invariance of analyte identity and complex mixtures.

As an example, biological pathogens, including biological threat agents, are living organisms that reproduce and sustain a population, which amplify, grow and re-infect, thereby resulting in an epidemic situation. The biological pathogens represent an extremely diverse range of microorganisms, which have no seemingly common attributes other than infecting the human and animal populations. The problem is therefore to detect and identify them at the earliest stage of invasion and at the lowest concentration.

Prior to DNA sequencing, the highest resolution techniques provided only protein and peptide-level structures as targets of analysis and assays. Many of the well-established protocols called for the examination of the size and shape of the pathogens along with the examination of the expressed proteins through biochemical and immunochemical assays. Advances in DNA sequencing technology have made it possible for scientists all over the world to sequence complete microbial genomes rapidly and efficiently. Access to the DNA sequences of entire microbial genomes has recently offered new opportunities to analyse and understand pathogens at the molecular level. Modern DNA sequencing techniques are able to detect pathogens in biological tissues and study variations in gene expression in response to the pathogenic invasion. These responses help in designing novel approaches for microbial pathogen detection and drug development. Identification of certain microbial pathogens as etiologic agents responsible for chronic diseases is leading to new treatments and prevention strategies for these diseases.

Majority of the modern chemical sensors used in pathogen detection are based upon the sequence-based recognition of DNA, structural recognition of pathogens or pathogen biomarkers, or cell-based function. However, the selection of the pathogen biomarkers introduces a serious challenge in the development of the sensors for detection of the biological pathogens. This is because most of the pathogen biomarkers have low selectivity and can distinguish between general classes of microorganisms, but are not able to identify the specific species or strain of organism. For example, calcium dipicolinate is a unique component of endospores. Dipicolinic acid can therefore be used to indicate the presence of endospores, but it cannot be able to distinguish between very dangerous Bacillus anthracis spores and other non-toxic Bacillus spores. The presence of the DNA as an additional indicator will be able to determine that the unknown material is biological in nature but will not be able to identify its source (unless extensive sequence-based analysis is used). Also cell metabolites are generally common to many different cell types and therefore extremely difficult to use for discrimination between specific microorganisms. In view of the above, there is a long-felt need for new methods and devices to detect and identify biological pathogens.

The use of the ultrasensitive and highly selective microelectronic sensors for the biological pathogen detection is the area that has not been developed yet. The reasons for that are many. Sensor arrays that detect multiple pathogen biomarkers produce a large number of false alarms because of their low selectivity. The concept of sensor arrays has been successfully used in the field of vapour analysis. In this approach each particular sensor of the sensor array was designed to respond to different properties of the vapours, followed by statistical methods to specifically identify the particular vapour from the fingerprint of the generated response from all the sensors of the array. However, since each pathogen species carries with it a unique DNA or RNA signature that differentiate it from other organisms, such approach cannot be effectively used for pathogen detection. In other words, each sensor of the array responds to different properties (biomarkers) of a pathogen. Therefore, such approach would require a well-characterised and already identified background signal to determine the fingerprints that would constitute a positive signal.

The ideal solution for a real-time sensing would be any specific response of a biological organism that results in instantaneous, specific and repeatable identification. However, as noted above, there are considerable technological and practical difficulties in the development of sensors that provide a real-time response for all three of these criteria. Immuno-assay techniques might give a similar specific analysis. However, their drawback, other than the long response time, is the requirement for special chemical consumables that add considerably to the logistic burden and costs. These can increase operational costs by hundreds of dollars per hour.

Optical technologies intrinsically result in real-time (bio)chemical detection. Sensors based on these technologies have been available to military and civil defense for quite some time. However, the common drawback of the optical sensors is low specificity. The sensors mostly offer a generic detection capability at best, since the optical similarity of the target particles with benign, naturally occurring backgrounds makes them difficult to distinguish. There are the some of the currently employed bio-agent detections strategies. Most represent a compromise between specificity, speed and cost.

Quantitative Polymerase Chain Reaction (qPCR) is capable of amplification and detection of a DNA sample from a single bio-agent cell within 30 minutes. Knowing the pathogen nucleic acid sequence makes it possible to construct oligoes for pathogen detection. These oligoes are at the basis of many highly specific analytical tests now on the market.

Microarray-based detection can combine powerful nucleic acid amplification strategies with the massive screening capability of microarray technology, resulting in a high level of sensitivity, specificity, and throughput. In addition to the previously mentioned caveats, the cost and organizational complexity of performing a large number of PCR reactions for downstream microarray applications render this option feasible but unattractive. This limitation has severely reduced the utility of this technique and impeded the continued development of downstream applications.

To sum up, the problem of accurate and reliable identification of pathogenic agents and their corresponding diseases is the weakest point in biological agent detection capability today. There is intense research for new molecular detection technologies that could be used for very accurate detection of pathogens that would be a concern to first responders. These include the need for ultrasensitive and highly selective sensors for biological pathogens detection in environmental, forensic and military applications. The benefits of specific (accurate) detection include saving millions of dollars annually by reducing disruption of the workforce and the national economy and improving delivery of correct protective countermeasures.

All said above regarding detection of biological pathogens also relate to the detection of other chemical and biological compounds, which may present threat or have medical reasons to be detected. The examples are many and may include explosives, toxins, DNA, proteins etc.

### Sub-THz and THz Spectroscopy

Recently it has become clear to scientists that sub-terahertz (sub-THz) and terahertz (THz) radiation could be extremely important for research related to the life sciences because of the unique capability of these low energy electromagnetic waves to interact with vibrations of atoms within biological molecules to produce specific molecular fingerprints (see for example, Globus et al. in “Terahertz Fourier transform characterization of biological materials in a liquid phase”, J. Physics D: Applied Physics, 39(15), 3405-3413). Sub-THz and THz spectroscopy uses wavelengths beyond those traditionally used for chemical and biomolecular analysis. Biological materials have found to be active in the frequency range of 30 GHz to 300 THz (the wavelength range, about 1 cm to 1 μm). These frequency and wavelength domains, the spectral range between the upper end of the radio frequencies and microwaves and the lowest optical frequencies were named the ‘Terahertz Gap’, because so little was known about them and because of the absence of radiation sources and detectors.

Sub-THz and THz vibrational spectroscopy is entirely based on the interaction of radiation in this particular frequency range with internal molecular vibrations of low energy. A majority of the sub-THz and THz experimental data have recently been reported on frequencies in this range and for relatively small biological molecules that are often prepared in crystalline form (for example, Heilweil et al. (2008), “Terahertz Spectroscopy of Biomolecules”, In Terahertz Spectroscopy, Taylor and Francis, London, 2008, Chapter 7, pp 269-297). Low-energy THz radiation interacts with the low-frequency internal molecular motions (vibrations) involving the weakest hydrogen bonds (H-bonds) and other weak connections within molecules by exciting these vibrations. The width of individual spectral lines and the intensity of resonance features, which are observed in sub-THz spectroscopy, are very sensitive to the relaxation processes of atomic dynamics (displacements) within a molecule. Those relaxation processes determine the discriminative capabilities of sub-THz spectroscopy. Appropriate spectral resolution must be used in THz spectroscopy to be able to acquire qualitative as well as quantitative information used to identify the molecules that will, in turn, increase detection accuracy and selectivity.

Because of their small size and relatively low absorption coefficient, the waves of the sub-THz and THz radiation easily propagate through any liquid, such as water, serum or any biological medium including the entire biological object, for example cells and skin. Safrai et al. (2012) in “The remote sensing of mental stress from the electromagnetic reflection coefficient of human skin in the sub-THz range”, Bioelectro-magnetics, 2012, 33(5), 375-82, and Safrai et al. (2014-1) in “Remote monitoring of phasic heart rate changes from the palm” in IEEE Transactions on Terahertz Science and Technology, 2014, 4, 618-624, reported that both physical and mental stress could be traced through the reflection coefficient of the hand, influenced by the activity of the sweat ducts, in the (75 GHz-110 GHz) and (110 GHz-170 GHz) frequency bands. The reflected signal was monitored from a distance of 72 cm using a Vector Network Analyser, while the patients' electrocardiograms (ECGs) were concurrently registered. Further, Safrai et al. (2014-2) in “The correlation of ECG parameters to the sub-THz reflection coefficient of human skin”, IEEE Transactions on Terahertz Science and Technology, 2014, 4(5), 624-630, reported on a good correlation between the reflection coefficient in the same frequency bands and some of the parameters of the ECG, mainly to the ST elevation.

### SUMMARY

The present disclosure describes embodiments of a method for chemical sensing and biomolecular diagnostics comprising:

(1) Applying a sample to be tested to a microelectronic senor;
(2) Recording electrical signals received from the sample with the microelectronic sensor in a form of a source-drain electric current of the microelectronic sensor over time (I_{DS} dynamics);
(3) Transmitting the recorded signals from said microelectronic sensor to an external memory for further processing; and
(4) Converting the transmitted signals to digital signals and processing the digital signals in the external memory, comparing said I_{DS} dynamics with negative control chemical or biomolecular I_{DS} waveforms stored in the external memory, and extracting chemical or biomolecular information from said waveforms in a form of readable data, thereby detecting and/or identifying a particular chemical or biological compound (target, analyte) in the sample and measuring its concentration;
characterised in that said microelectronic sensor comprises at least one open-gate pseudo-conductive high-electron mobility transistor for amplifying signals in the frequency range of 30 GHz to 300 THz, said transistor comprising:
(i) a multilayer heterojunction structure being composed of III-V single-crystalline or poly-crystalline semiconductor materials and deposited on a substrate layer or placed on free-standing membranes, said structure comprising at least one buffer layer and at least one barrier layer, said layers being stacked alternately;
(ii) a conducting channel comprising a two-dimensional electron gas (2DEG) or a two-dimensional hole gas (2DHG), formed at the interface between said buffer layer and said barrier layer, and upon applying a bias to said transistor, capable of providing electron or hole current, respectively, in said transistor between source and drain contacts;
(iii) the source and drain contacts connected to said 2DEG or 2DHG conducting channel and to electrical metallisations for connecting said transistor to an electric circuit; and
(iv) a Vivaldi antenna electrode placed on the top layer between said source and drain contact areas in an open gate area of the transistor and capable of detecting electrical signals in the frequency range of 30 GHz to 300 THz;
said transistor is characterised in that the thickness (d) of the top layer of said heterojunction structure in the open gate area is 5-9 nanometres (nm) which corresponds to the pseudo-conducting current range between normally-on and normally-off operation mode of the transistor, and the surface of said top layer has a roughness of about 0.2 nm or less, wherein the combination of said thickness and said roughness of the top layer allows to observe the pseudo-conducting current in said transistor.

In some embodiments, said transistor further comprises at least one molecular or bio-molecular layer immobilised within the open gate area of said transistor and capable of binding or adsorbing target (analyte) gases, chemical compounds or biomolecules from the environment. In another embodiment, the transistor is not coated with a molecular or biomolecular layer and is capable of remotely detecting target (analyte) gases, chemical compounds or biomolecules from the environment.

In a further embodiment, the source and drain contacts of said transistor are ohmic. In a particular embodiment, the electrical metallisations of said transistor are capacitively-coupled to said 2DEG or 2DHG conducting channel for inducing displacement currents, thus resulting in said source and drain contacts being non-ohmic. In this case, since the source and drain contacts are non-ohmic, the DC readout cannot be done. Instead, to electrically contact the 2DEG/2DHG channel underneath, about 5-20 nm bellow the metallisations, the AC readout or impedance measurements of the electric current flowing through the 2DEG/2DHG-channel must be performed. In this case, the capacitive coupling of the non-ohmic metal contacts with the 2DEG/2DHG channel is normally induced at the frequency higher than 30 kHz.

In a particular embodiment, said transistor further comprises a dielectric layer deposited on top of said multilayer hetero-junction structure. In another particular embodiment, the thickness of the top layer recessed in the open gate area of said transistor is 6-7 nm, more specifically 6.2 nm to 6.4 nm. The surface roughness of the top layer recessed in the open gate area of said transistor is specifically 0.1 nm or less, more specifically 0.05 nm or less.

Non-limiting examples of the molecular or biomolecular layer of said transistor is a cyclodextrin, 2,2,3,3-tetrafluoropropyloxy-substituted phthalocyanine or their derivatives, or said molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

In a further embodiment, said microelectronic sensor is suitable for detection and continuous monitoring of electrical signals in the frequency range of 30 GHz to 300 THz and consequently, for chemical sensing and biomolecular diagnostics in said frequency range, said sensor having a remote readout and comprising:

(a) at least one said transistor (100);
(b) an integrated circuit (101) for storing and processing a signal in a sub-THz or THz frequency domain, and for modulating and demodulating a radio-frequency (RF) signals;
(c) an μ-pulse generator (102) for pulsed RF signal generation;
(d) an integrated DC-RF current amplifier or lock-in amplifier (103) connected to said μ-pulse generator (102) for amplification of the signal obtained from said μ-pulse generator;
(e) an analogue-to-digital converter (ADC) (104) with in-built digital input/output card connected to the amplifier (103) for converting the received analogue signal to a digital signal and outputting said digital signal to a microcontroller unit;
(f) the microcontroller unit (MCU) (105) for processing and converting the received digital signal into data readable in a user interface or external memory; and
(g) a wireless connection module (106) for wireless connection of said microelectronic sensor to said user interface or external memory.

In yet further embodiment, said microelectronic sensor is suitable for detection and continuous monitoring of electrical signals in the frequency range of 30 GHz to 300 THz and consequently, for chemical sensing and biomolecular diagnostics in said frequency range, said sensor having a remote readout and comprising:

(a) an array (110) of said transistors (100), wherein each transistor (100) in said array (110) has an integrated Vivaldi antenna and connected to its dedicated electrical contact line;
(b) a row multiplexer (107) connected to said array (110) for addressing a plurality of said transistors (100) arranged in rows, selecting one of several analogue or digital input signals and forwarding the selected input into a single line;
(c) a column multiplexer (108) connected to said array (110) for addressing a plurality of said transistors (100) arranged in columns, selecting one of several analogue or digital input signals and forwarding the selected input into a single line;
(d) an integrated circuit for storing and processing said signals in a sub-THz or THz frequency domain, and for modulating and demodulating a radio-frequency (RF) signals;
(e) an μ-pulse generator (102) for pulsed RF signal generation;
(f) an integrated DC-RF current amplifier or lock-in amplifier (103) connected to said μ-pulse generator (102) for amplification of the signal obtained from said μ-pulse generator;
(g) an analogue-to-digital converter (ADC) (104) with in-built digital input/output card connected to the amplifier (103) for converting the received analogue signal to a digital signal and outputting said digital signal to a microcontroller unit;
(h) the microcontroller unit (MCU) (105) for processing and converting the received digital signal into data readable in a user interface or external memory; and
(i) a wireless connection module (106) for wireless connection of said microelectronic sensor to said user interface or external memory.

The sample can be either in a gas phase or in a liquid phase. Non-limiting examples of the chemicals to be tested are a toxic metal, such as chromium, cadmium or lead, a regulated ozone-depleting chlorinated hydrocarbon, a food toxin, such as aflatoxin, or shellfish poisoning toxin, such as saxitoxin or microcystin, a neurotoxic compound, such as methanol, manganese glutamate, nitrix oxide, Botox, tetanus toxin or tetrodotoxin, oxybenzone, Bisphenol A, or butylated hydroxyanisole, an explosive, such as picrate, nitrate, trinitro derivative, such as 2,4,6-trinitrotoluene (TNT), 1,3,5-trinitro-1,3,5-triazinane (RDX), trinitroglycerine, N-methyl-N-(2,4,6-trinitrophenyl)nitramide (nitramine or tetryl), pentaerythritol tetranitrate (PETN), nitric ester, azide, derivate of chloric and perchloric acids, fulminate, acetylide, and nitrogen rich compound, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosive, such as a decomposition gas or taggant.

Non-limiting examples of the biological compounds to be detected area a biological pathogen, such as a respiratory viral or bacterial pathogen, an airborne pathogen, a plant pathogen, a pathogen from infected animals or a human viral pathogen.

Various embodiments may allow various benefits, and may be used in conjunction with various applications. The details of one or more embodiments are set forth in the accompanying figures and the description below. Other features, objects and advantages of the described techniques will be apparent from the description and drawings and from the claims

### BRIEF DESCRIPTION OF THE DRAWINGS

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIGS. 1a-1c schematically show the quantum well at three different biasing conditions:

FIG. 1a: positive gate potential (+VG) is much higher than threshold voltage (VT),

FIG. 1b: 0V gate potential, and

FIG. 1c: negative gate potential (−VG) is below threshold voltage (VT).

FIG. 2 schematically shows a cross-sectional (XZ) view (A-A) of the PC-HEMT of an embodiment.

FIG. 3 schematically shows the top (XY) view and the basic topology of the sensor of an embodiment.

FIG. 4a schematically shows the dependence of the source-drain current (a charge carrier density) induced inside the 2DEG channel of a GaN/AlGaN HEMT on the thickness of the AlGaN layer recessed in the open gate area.

FIG. 4b illustrates a theory behind the 2DEG formation (charge neutrality combined with the lowest energy level) at the conduction band discontinuity.

FIG. 5a schematically shows the 2DEG area created in the step of the 2DEG-pattering via ion implantation during the manufacturing process. AZ 4533 is a positive thick resist.

FIG. 5b shows the lithographic mask of the sensor layout of the present invention.

FIG. 5c shows the lithographic image of the 2DEG channel formed with AZ 4533 thick resist lithography over the mask shown in FIG. 5b.

FIGS. 5d-5e show the mask and the corresponding lithographic image, respectively, of the sensor layout of the present invention.

FIG. 5f shows the ±2-μm alignment precision on 25×25 mm2 samples in the lithography of the sensor layout of the present invention.

FIG. 5g shows the lithographic images of the multichannel samples.

FIG. 5h shows the fixed sample on the Si—GaN/AlGaN wafer prepared for ion implantation and containing around 30-32 sensors with 4-8 channels on each sample.

FIG. 5i shows the lithographic image of the sensor layout with the AZ4533 resist after development, prepared for ion implantation.

FIG. 5j shows the 2DEG channels (dark) patterned by ion-implantation after the resist removal.

FIG. 5k shows the visible non-implanted area containing the conductive 2DEG channel.

FIG. 6a shows the AFM surface image of the top recessed layer of the PC-HEMT made by the manufacturing process of the present invention. The measured RMS value of the surface roughness is 0.674 nm in this case.

FIG. 6b shows the AFM surface image of the top recessed layer of the HEMT made by a conventional manufacturing process. The measured RMS value of the surface roughness is 1.211 nm in this case.

FIG. 6c shows the time-dependent plot of the drain-source electric current I_{DS} of the nitrogen oxide sensor of the present invention measuring 100 ppb of the NO₂ gas in humid air, where the sensor is based on the PC-HEMT made by the manufacturing process of the present invention.

FIG. 6d shows the time-dependent plot of the drain-source electric current I_{DS} of the nitrogen oxide sensor measuring 100 ppb of the NO₂ gas in humid air, where the sensor is based on the HEMT made by a conventional manufacturing process.

FIG. 7a schematically shows the formation of the 2DEG and 2DHG conducting channels in the Ga-face three-layer Ga/AlGaN/GaN PC-HEMT structure.

FIG. 7b schematically shows the formation of the 2DEG and 2DHG conducting channels in the N-face three-layer Ga/AlGaN/GaN PC-HEMT structure.

FIG. 8 schematically shows the formation of the 2DEG conducting channel in the N-face three-layer GaN/AlGaN/GaN PC-HEMT structure with an ultrathin AlN or AlGaN barrier layer with high Al-content on top of the 2DEG channel for improved confinement.

FIG. 9 shows the model of a Vivaldi antenna realised on a thin dielectric substrate. An exponential function is used for the taper profile. The entire domain is bounded by a perfectly matched layer.

FIG. 10 shows the electric field distribution in the Vivaldi antenna plane at 480 GHz.

FIG. 11a shows the far-field directional radiation pattern of the Vivaldi antenna starting from 240 GHz till 780 GHz.

FIG. 11b shows the corresponding 3D far-field pattern at 480 GHz.

FIG. 12 shows the exemplary two-dimensional photonic crystals (a=160 μm, d=60 μm) deposited on top of the metal layer of the Vivaldi antenna.

FIG. 13 shows the position of the metal connector on the Vivaldi antenna for coupling with the PC-HEMT of an embodiment.

FIG. 14a illustrates the barrier layer/liquid or gas interface with the double layer formation, simplified equivalent interface circuity and ion electrodynamics during exposure of the sensor to a positive charge.

FIG. 14b illustrates the barrier layer/liquid or gas interface with the double layer formation, simplified equivalent interface circuity and ion electrodynamics during exposure of the sensor to a negative charge.

FIG. 15 schematically shows a microelectronic sensor comprising a single PC-HEMT of the embodiments with the integrated Vivaldi antenna, for detection and continuous monitoring of electrical signals in sub-THz and THz frequency ranges, with a remote readout.

FIG. 16 schematically shows a microelectronic sensor comprising an array of the PC-HEMTs of the embodiments with the integrated Vivaldi antennas, for detection and continuous monitoring of electrical signals in sub-THz and THz frequency ranges, with a remote readout.

FIGS. 17a-17b show the exemplary sensor layout of the present invention.

FIG. 18a schematically show the experimental sensor setup with the sensor of the present invention.

FIGS. 18b-18c show the photographs of the experimental sensor setup in the lab.

FIG. 19a shows the results of the first experiment with Sensor A (its I_{DS} dynamics) upon introducing a drop of the infected blood serum onto the surface of the sensor. The four curves shown in the figure represent four serum samples having different concentrations of Lime borreliosis in the concentration range up to 100 pg/ml, while the fifth (bold) curve shows the average of these four tests.

FIG. 19b shows the results of the first experiment with Sensor B (its I_{DS} dynamics) upon introducing a drop of the negative control serum onto the surface of the sensor, where Sensor B was used as a negative control receiving a drop of a clean human serum without the infection. The three curves shown in the figure represent three different samples of a clean human serum, while the fourth (bold) curve shows the average of these three tests.

FIG. 19c shows the comparison of the average curves for Sensors A and B in the first experiment.

FIG. 19d shows the normalised comparison of Sensors A and B in the first experiment, with graphical calculation of response rate and kinetics of Sensor A toward the infection.

FIG. 20a shows the I_{DS} dynamics of the Sensors A and B in the second experiment with the p-Tau protein. Sensor A was exposed to positive p-Tau protein serum samples with four different concentrations in the concentration range up to 100 pg/ml, while the fifth (bold) curve shows the average of these four tests.

FIG. 20b shows the results of the second experiment with Sensor B (its I_{DS} dynamics) that was used as a negative control receiving a drop of human blood serum with the concentration of the p-Tau protein 0.05 pg/ml in the control sample. The five curves shown in the figure represent five different samples of a clean serum, while the sixth (bold) curve shows the average of these five tests.

FIG. 20c shows the comparison of the average curves for Sensors A and B in the second experiment.

FIG. 20d shows the normalised comparison of Sensors A and B in the second experiment, with graphical calculation of response rate and kinetics of Sensor A toward the presence of the protein.

FIG. 21 shows the results of the third experiment with Sensors A and B in the form of the measured I_{DS} dynamics. A peanut sample was introduced onto the surface of Sensor A for positive measurements, where Sensor B was used as a negative control. Each curve shown in the figure is an average of three independent tests. The curves were normalised to baseline for comparison.

FIG. 22 shows the results of the fourth experiment with Sensors A and B in the form of the I_{DS} dynamics. An anti-centromere sample was dropped onto the surface of Sensor A for positive measurements, where Sensor B was used as a negative control. Each curve shown in the figure is an average of two independent tests. The curves were normalised to baseline for comparison.

FIG. 23a illustrates an equivalent circuit of the PC-HEMT-based sensor of the present invention having the molecular or biomolecular layer immobilised on its surface.

FIG. 23b shows a simplified equivalent circuit of the PC-HEMT-based sensor of the present invention having the molecular or biomolecular layer immobilised on its surface.

FIG. 23c displays the theoretical transistor-transfer function in the most simplified form.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

The polarization doped high-electron-mobility transistor (HEMT) is a field effect transistor (FET) in which two layers of different bandgap and polarisation field are grown upon each other forming the heterojunction structure. This transistor is essentially based on at least two layers of III-V semiconductor materials, such as gallium nitride (GaN) and aluminium gallium nitride (AlGaN). As a consequence of the discontinuity in the polarisation field, surface charges are created at the interface between the layers. If the induced surface charge is positive, electrons will tend to compensate the induced charge resulting in the formation of the channel. Since the channel electrons are confined in a quantum well in an infinitely narrow spatial region at the interface between the layers, these electrons are referred to as a two-dimensional electron gas (2DEG). This special confinement of the channel electrons in the quantum well actually grants them two-dimensional features, which strongly enhance their mobility surpassing the bulk mobility of the material in which the electrons are flowing.

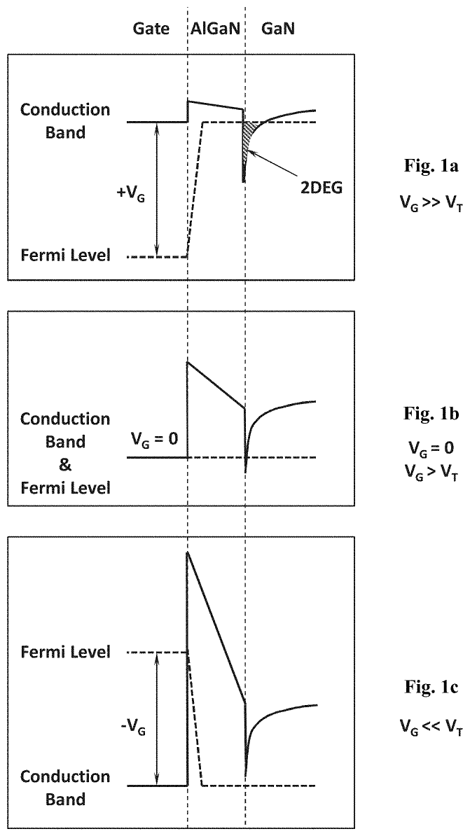

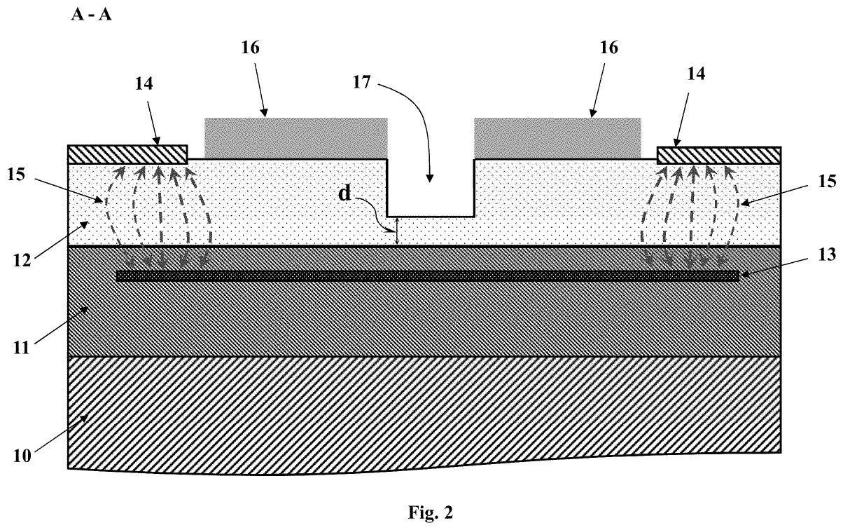

FIGS. 1a-1c schematically shows the quantum well at three different biasing conditions starting from the positive gate potential (V_{G}), much higher than the threshold voltage (V_{T}), and going down to the 0V gate potential and further to the negative values below the threshold voltage. The V_{T} is defined as a voltage, which is required to populate electrons at the interface between the GaN layer and the AlGaN layers, thereby creating conductivity of the 2DEG channel. Since the 2DEG channel electrons occupy energy levels below the Fermi level, the Fermi level in a quantum well is located above several energy levels when V_{G}>>V_{T} (FIG. 1a). This enables high population of channel electrons and consequently, high conductivity. The 2DEG channel is turned on in this case. However, when V_{G} decreases to 0V (FIG. 1b), the Fermi level also drops with respect to the quantum well. As a result, much fewer electron energy levels are populated and the amount of the 2DEG channel electrons significantly decreases. When V_{G} much less than V_{T} (FIG. 1c), all electron energy levels are above the Fermi level, and there is no the 2DEG electrons below the gate. This situation is called “channel depletion”, and the channel is turned off.

Many commercially available HEMTs based on the layers of III-V semi-conductor materials have a negative value of V_{T}, resulting in a “normally-on” operation mode at 0V gate potential. They are called “depletion-mode” semiconductor transistors and used in various power switching applications when the negative voltage must be applied on the gate in order to block the current. However, for safe operation at high voltage or high power density, in order to reduce the circuit complexity and eliminate standby power consumption, the transistors with “normally-off” characteristics are preferred. The high voltages and high switching speeds allow smaller, more efficient devices, such as home appliances, communications and automobiles to be manufactured. To control the density of electrons in the 2DEG channel and to switch the HEMT on and off, the voltage at the gate of the transistor is normally regulated.

Several techniques to manufacture the normally-off semiconductor structures have been reported. Burnham et al (2010) proposed normally-off structures of the recessed gate type. In this structure, the AlGaN barrier layer is etched and the gate is brought closer to the interface between the AlGaN barrier layer and the GaN buffer layer. As the gate approaches the interface between the layers, the VT increases. Thus, the normally-off operation of the 2DEG conducting channel is achieved once the depletion region reaches the interface and depletes the 2DEG channel at zero gate voltage. The major advantages of these structures are relatively lower power consumption, lower noise and simpler drive circuits. They are currently used, for example, in microwave and millimetre wave communications, imaging and radars.

Chang et al (2009) proposed instead of etching the relatively thick barrier layer to approach the AlGaN/GaN interface, to use a very thin AlGaN barrier. This structure also achieves the normally-off operation of the 2DEG channel by approaching the gate towards the AlGaN/GaN interface. Chen et al (2010) proposed to use the fluorine-based plasma treatment method. Although many publications have adopted various methods to achieve normally-off devices with minimum impact on the drain current, they unfortunately sacrificed device turn-on performance.

The present application describes embodiments of a microelectronic sensor based on a combination of an open-gate pseudo-conducting high-electron mobility transistor (PC-HEMT) and Vivaldi antenna installed in the open gate area of the transistor.

As shown in FIG. 2, the PC-HEMT of the present application, capable of sensing sub-THz and THz radiation from any gas, liquid or solid medium, comprises:

(a) a multilayer heterojunction structure composed of III-V single-crystalline or poly-crystalline semiconductor materials, said structure comprising at least one buffer layer (11) and at least one barrier layer (12), said layers being stacked alternately, and said structure being deposited on a substrate layer (10);
(b) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current in the transistor between source and drain contact areas (15);
(c) electrical metallisations (14) capacitively-coupled to said 2DEG or 2DHG conducting channel (13) via the source and drain contact areas (15) for connecting said transistor to an electric circuit; and
(d) a Vivaldi antenna electrode (16) placed on a top layer between said source and drain contact areas (15) in an open gate area of the transistor and capable of detecting electrical signals in the sub-THz- and THz-frequency range of 30 GHz to 300 THz (the corresponding wavelength range is about 1 cm to 1 μm).

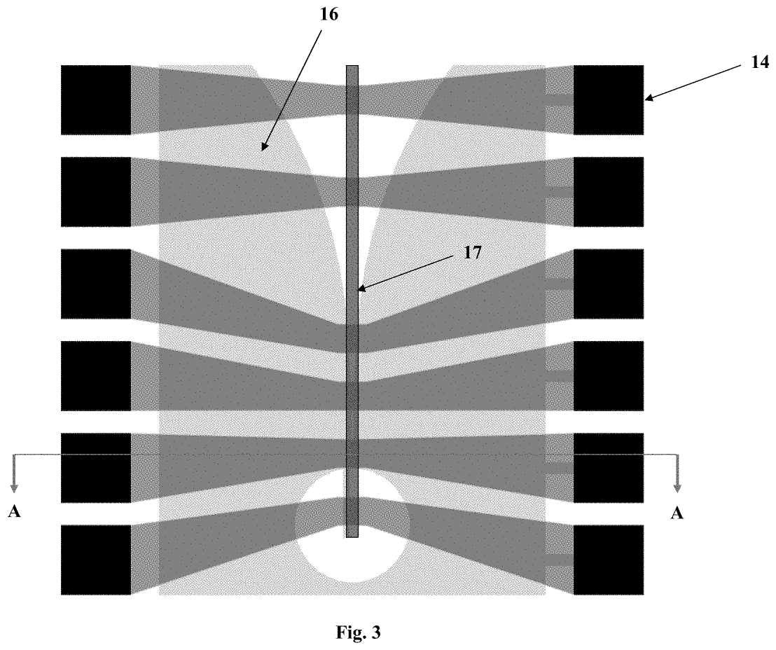

The functional basic topology of the microelectronic sensor of the present embodiments is schematically shown in FIG. 3. The sensor detection principle is based on the field effect of electric current modulation in a DC-mode within the 2DEG/2DHG conducting channel (13) achieved by Vivaldi-shaped gate antenna (16). In fact, the Vivaldi-shaped gate antenna (16) is capable of strongly concentrating the electric field strength of sub-THz and THz radiation in a very small area above the ultra-charge-sensitive 2DEG/2DHG channel, thereby affecting its conductivity in the DC-mode by accumulation effect. Moreover, the S11-S12 parameters of the PC-HEMT of the present embodiments may also be measured at radio frequencies (RF) of 1 to 60 GHz using the ‘beating’ effect of sub-THz radiation.

The transistor shown in FIGS. 2 and 3 may further comprise a dielectric layer of 1-10 nm thickness. This dielectric layer may be deposited on top of the barrier layer (12). The Vivaldi antenna gate electrode, made for example from gold, is then placed directly on the dielectric layer. This configuration prevents strong electrical leakage at the metal/top layer interface. The dielectric layer used for the device passivation, may be made, for example, of SiO—SiN—SiO (“ONO”) stack of 100-100-100 nm thickness or SiN—SiO—SiN (“NON”) stack having the same thicknesses. It may be deposited on top of the barrier layer by a method of plasma-enhanced chemical vapour deposition (PECVD), which is a stress-free deposition technique.

The electrical metallisations (14) connect the transistor to the electric circuit and allow the electric current to flow between non-ohmic contact areas (15) via the two-dimensional electron gas (2DEG) or two-dimensional hole gas (2DHG) channel (13). The metallisations (14) may be made of metal stacks, such as Cr/Au, Ti/Au, Ti/W, Cr/Al and Ti/Al. The Cr or Ti layers of the metal stack is, for example, of 5-10 nm thickness, while the second metal layer, such as Au, W and Al, is of 100-400 nm thickness. The metallisations (14) may be chosen according to the established technology and assembly line at a particular clean room fabrication facility.

In some embodiments, substrate layer (10) may be composed, for example, of sapphire, silicon, silicon carbide, gallium nitride or aluminium nitride. The hetero junction structure (11, 12) may be deposited on the substrate layer (10), for example, by a method of metalorganic chemical vapour deposition (MOCVD), thereby forming the pseudo-conducting 2DEG or 2DHG channel (13) in the close proximity to the interface between the buffer layer (11) and the barrier layer (12). The barrier layer (12) then may be either recessed or grown in the recessed open-gate area (17) as a very thin layer between the source and drain contact areas (15).

The 2DEG or 2DHG channel (13) formed near the interface between the buffer layer (11) and the barrier layer (12) serves as a main sensitive element of the transistor reacting to a surface charge and potential. The 2DEG or 2DHG channel (13) is configured to interact with very small variations in surface or proximal charge or changes of electrical field on the top layer/Vivaldi antenna gate interface interacting with the donor-like surface trap states of the barrier layer. This will be discussed below in detail.

The term “2DEG” mentioned in the present description and claims should not be understood or interpreted as being restricted to the two-dimensional electron gas. As stated above and will be explained later in this application, the two-dimensional hole gas may also be a possible current carrier in a specific heterojunction structure. Therefore, the term “2DEG” may be equally replaced with the term “2DHG” without reference to any particular PC-HEMT configuration.

In some embodiments, the PC-HEMT multilayer heterojunction structure of the embodiments is grown from any available III-V single-crystalline or polycrystalline semiconductor materials, such as GaN/AlGaN, GaN/AlN, GaN/InAlGaN, GaN/InAlN, GaN/InN, GaAs/AlGaAs, InN/InAlN or LaAlO₃/SrTiO₃. In a specific case of the hetero-junction structure grown from GaN/AlGaN, it has been experimentally found that the highest sensitivity of the sensor is achieved when thickness of the top recessed layer (GaN or AlGaN) in the open gate area between the source and drain contacts is 5-9 nm, preferably 6-7 nm, more preferably 6.2-6.4 nm. In addition, it was also found that the sensor exhibits its highest sensitivity when surface roughness of the top recessed layer is about 0.2 nm or less, preferably 0.1 nm or less, more preferably 0.05 nm.

Thus, the top layer recessed in the open gate area to 5-9 nm must be optimised for significantly enhancing sensitivity of the sensor. This specific thickness of the top layer was surprisingly found to correspond to the “pseudo-conducting” current range between normally-on and normally-off operation modes of the 2DEG channel and requires further explanation.

“Pseudo-contacting” (to distinguish from normally-conducting) current range of the 2DEG channel is defined as an operation range of the channel between its normally-on and normally-off operation modes. “Trap states” are states in the band-gap of a semiconductor which trap a carrier until it recombines. “Surface states” are states caused by surface reconstruction of the local crystal due to surface tension caused by some crystal defects, dislocations, or the presence of impurities. Such surface reconstruction often creates “surface trap states” corresponding to a surface recombination velocity.

Classification of the surface trap states depends on the relative position of their energy level inside the band gap. The surface trap states with energy above the Fermi level are acceptor-like, attaining negative charge when occupied. However, the surface trap states with energy below the Fermi level are donor-like, positively charged when empty and neutral when occupied. These donor-like surface trap states are considered to be the source of electrons in the formation of the 2DEG channel. They may possess a wide distribution of ionization energies within the band gap and are caused by redox reactions, dangling bonds and vacancies in the surface layer. A balance always exists between the 2DEG channel density and the number of ionised surface donors which is governed by charge neutrality and continuity of the electric field at the interfaces.

Thus, the donor-like surface traps at the surface of the top layer are one of the most important sources of the 2DEG in the channel. However, this only applies for a specific top layer thickness. In a relatively thin top layer, the surface trap state is below the Fermi level. However, as the top layer thickness increases, the energy of the surface trap state approaches the Fermi energy until it coincides with it. The thickness of the top layer corresponding to such situation is defined as “critical”. At this point, electrons filling the surface trap state are pulled to the channel by the strong polarisation-induced electric field found in the top layer to form the 2DEG instantly.

If the surface trap states are completely depleted, further increase in the top layer thickness will not increase the 2DEG density. Actually, if the 2DEG channel layer fails to stretch the top layer, the later will simply relax. Upon relaxation of the top layer, many crystal defects are created at the interface between the top layer and the layer right underneath it, and the piezoelectric polarisation instantly disappears causing deterioration in the 2DEG density.

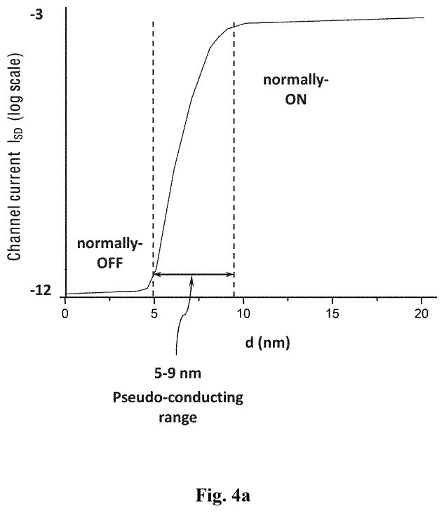

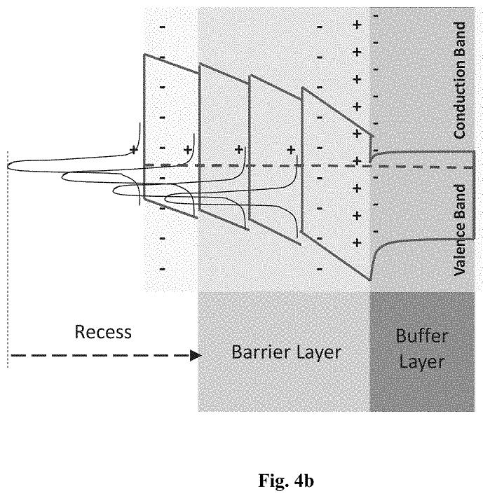

In order to illustrate the above phenomenon of the pseudo-conducting current, reference is now made to the following figures. FIG. 4a shows the dependence of the source-drain current (a charge carrier density) on the recessed AlGaN layer thickness. As seen from the plot, transistors that have a thickness of the top layer larger than 9 nm form normally-on 2DEG channels. In such transistors, due to the inherent polarisation effects present in the III-V materials, a thin sheet of charges is induced at the top and bottom of the interfaces of the top layer. As a result, a high electric field is induced in the top layer, and surface donor states at the top interface start donating electrons to form the 2DEG channel at the proximity of the hetero-junction interface without the application of a gate bias. These transistors therefore constitute normally-on devices. On the other hand, the transistors that have a thickness of the top layer lower than about 5 nm act constitute normally-off devices. Energy equilibrium between the donor surface trap states and AlGaN tunnel barrier leads to the 2DEG formation (charge neutrality combined with the lowest energy level) at the conduction band discontinuity. As explained above, decrease in the thickness of the AlGaN layer results in increase of the energy barrier. As a result, the ionisable donor-like surface trap states, which are responsible for electron tunnelling from the surface to 2DEG, drift bellow the Fermi level, thereby minimizing the electron supply to the 2DEG channel. This theoretical situation is further illustrated in FIG. 4b. Therefore, the recess of the AlGaN layer from 9 nm to 5 nm leads to huge drop in conductivity of the two-dimensional electron gas for six orders of magnitude.

In view of the above, it is clear that the mechanism of the 2DEG depletion based on recessing the top layer is strongly dependent on the donor-like surface trap states (or total surface charge). As the thickness of the top layer decreases, much less additional external charge is needed to apply to the top layer surface in order to deplete the conductive 2DEG channel. There is a critical (smallest) barrier thickness, when the this channel is mostly depleted but still highly conductive due to a combination of the energy barrier and the donor surface trap states energy. At this critical thickness, even the smallest energy shift at the surface via any external influence, for example polarisation of the surface, leads immediately to the very strong 2DEG depletion. As a result, the surface of the top layer at this critical thickness is extremely sensitive to any smallest change in the electrical field of the surroundings. Thus, the recess of the top layer from 9 nm down to 5 nm significantly reduces the 2DEG density, brought the sensor of the invention to the “near threshold” operation and results in highly increased surface charge sensitivity. The specific 5-9 nm thickness of the top layer is actually responsible for the pseudo-conducting behaviour of the 2DEG channel and gives the sensor an incredible sensitivity.

The top layer is recessed to this specific thickness after subjecting to short plasma activation by an ultra-low damage reactive-ion etching technique using inductively-coupled plasma (ICP) with a narrow plasma-ion energy distribution. Such short plasma treatment allows much lower roughness of the surface, which is a function of the semiconductor vertical damage depth during the plasma etching process. Such low surface roughness (about 0.2 nm and less) can be achieved only via this ICP-RIE ultra low damage etching process with a narrow plasma-ion energy distribution, and this inherently results in a very low vertical damage depth to the top layer, which allows the minimal surface scattering and minimal surface states-2DEG channel interaction with the maximum signal-to-noise ratio of the sensor. Thus, the depth effect of the vertical sub-nanometre damage to the top recessed layer, due to an ultra-low damage ICP-RIE etching process with a very narrow plasma-ion energy distribution, is the only way to optimally achieve the required sub-nanometre roughness of the semiconductor surface. This inherently results in an adjustable pseudo-conductive working point with the highest charge sensitivity ever possible. This depth effect is always inherent to the sub-nanometre roughness of the semiconductor surface, which was measured using AFM (atomic force microscope).

Thus, in addition to the recessed top layer thickness, roughness of the top layer surface is another very important parameter that has not been previously disclosed. It has been surprisingly found that the roughness of the top layer surface (in the open gate sensitive area) bellow 0.2 nm prevents scattering of the donor-like surface trap states. Thus, combination of these two features: 5-9 nm thickness of the top layer in the open gate area and strongly reduced roughness of its surface (bellow 0.2 nm) make the sensor incredibly sensitive.

In a certain aspect, the method for manufacturing of the PC-HEMTs of the present invention comprises the following steps:

Step 1: Plasma-enhanced atomic layer deposition (ALD) of alumina (Al₂O₃) on a pre-aligned masked Si—GaN/AlGaN wafer with nitrogen-plasma de-trapping for the thickness of the Al₂O₃ layer being 3-10 nm. The Al₂O₃ layer thickness was measured with an X-ray reflectometer.
Step 2: Plasma-enhanced atomic layer deposition (ALD) pattering of the wafer coated with the thin Al₂O₃ layer in Step 1, with hydrogen fluoride (HF) or using the aforementioned reactive-ion etching (RIE) technique.
Step 3: Optionally creating the source and drain ohmic contacts (in case ohmic contacts are required) on the coated wafer obtained in Step 2 from metal stacks, for example Ti/Al/Mo/Au, Ti/Al/Ni/Au, Ti/Au and Ti/W, having 15-50 nm thickness, using spin-coating technique or e-beam physical vapour deposition (VPD) of the stack metals. The deposition rates using the e-VPD technique were determined for the ohmic-stack metals using the Dektak Profilometer with dummy lift-off samples.
Step 4: Two-dimensional electron gas (2DEG) channel-pattering of the wafer obtained in Step 3 with argon- or nitrogen-ion implantation.
Step 5: Plasma-enhanced chemical vapour deposition (CVD) of the ONO stack over the wafer obtained in Step 4. This is the stress-free technique to deposit the layer of the SiO—SiN—SiO stack having an exemplary thickness of about 200-300 nm and structured by the ICP-RIE dry etching, which is the CF4-based etching method. In this step, the pseudo-conducting channel areas and ohmic electrical contact pads of the transistor become available.
Step 6: Optional lift-off deposition of an Au or Ti/W-CMOS-gate electrode (in case a gate electrode is to be deposited on the top layer of the heterojunction structure for an integrated MMIC-HEMT-based amplifier manufacturing).
Step 7: Optional plasma-enhanced ALD pattering with RIE or HF above sensing area (in case the plasma-enhanced ALD layer deposited in Step 1 is removed separately to ONO stack).
Step 8: Atomic layer etching (ALE) of the wafer obtained in Steps 5-7. This sophisticated technique carried out in the clean manufacturing cluster of the applicant is the only technique allowing the removal of individual atomic layers (the top atomic layers of the wafer). ALE is a way better-controlled technique than RIE, though it has not been commercially used until now because very sophisticated gas handling is required, and removal rates of one atomic layer per second are the real state of the art. This step is the step of creating the pseudo-conducting working point of the transistor, because ALE allows achieving the specific thickness of 5-9 nm thickness of the top layer in the open gate area with the extremely low surface roughness of the top layer below 0.2 nm.
Step 9: Optional plasma-enhanced CVD or ALD of the dielectric layer used for device passivation and in some gas sensors.
Step 10: Optional deep reactive-ion etching (DRIE or Bosch process) of the Si-substrate under sensing areas (in case the substrate is on the free-standing membranes—used, for example, in RF-HEMTs, FBAR and SAW sensors).

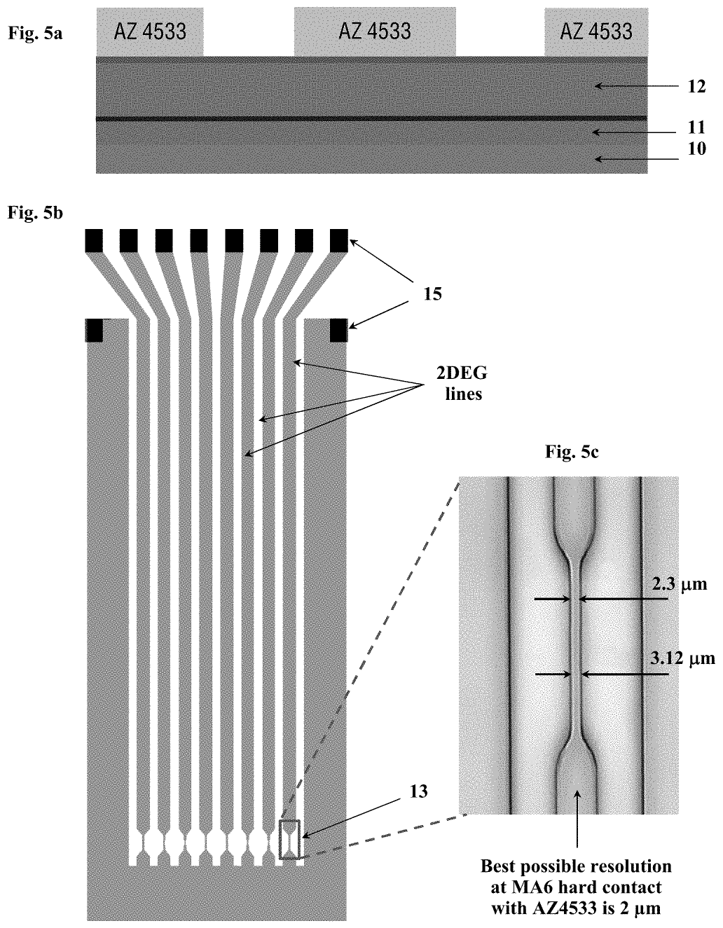

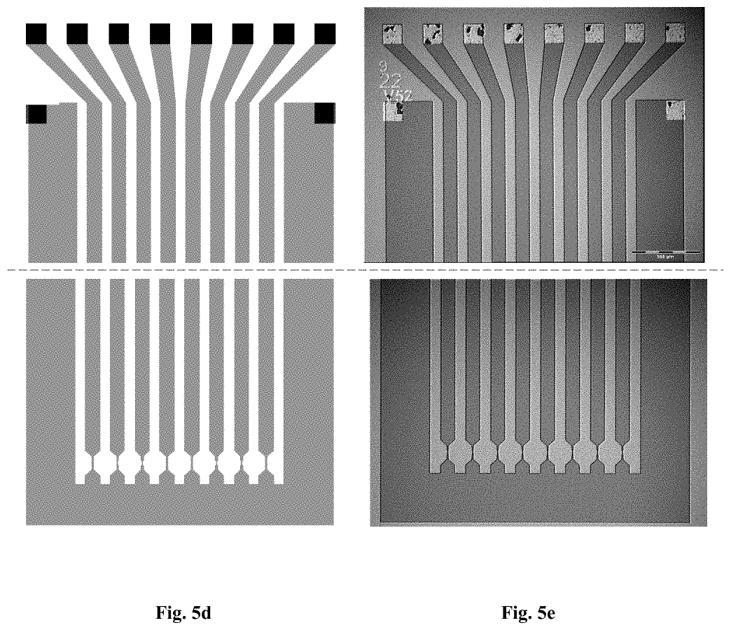

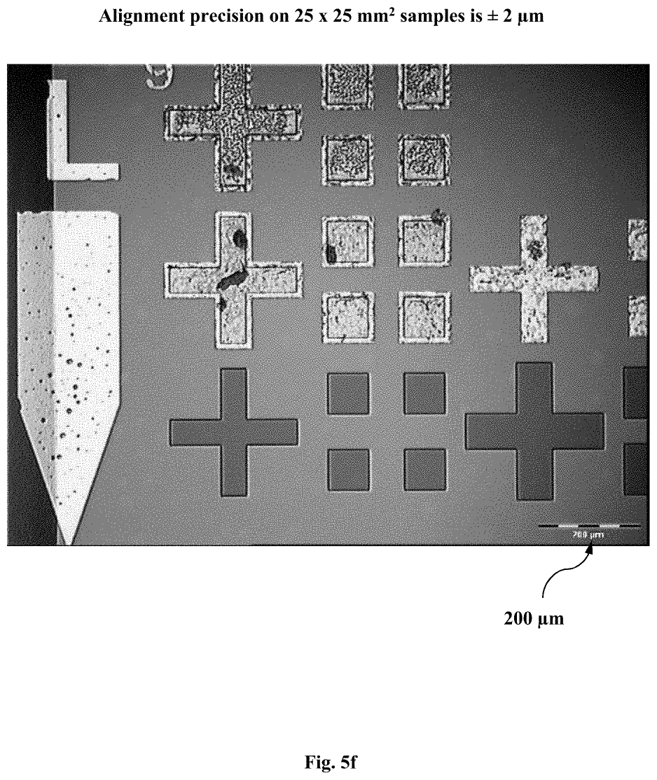

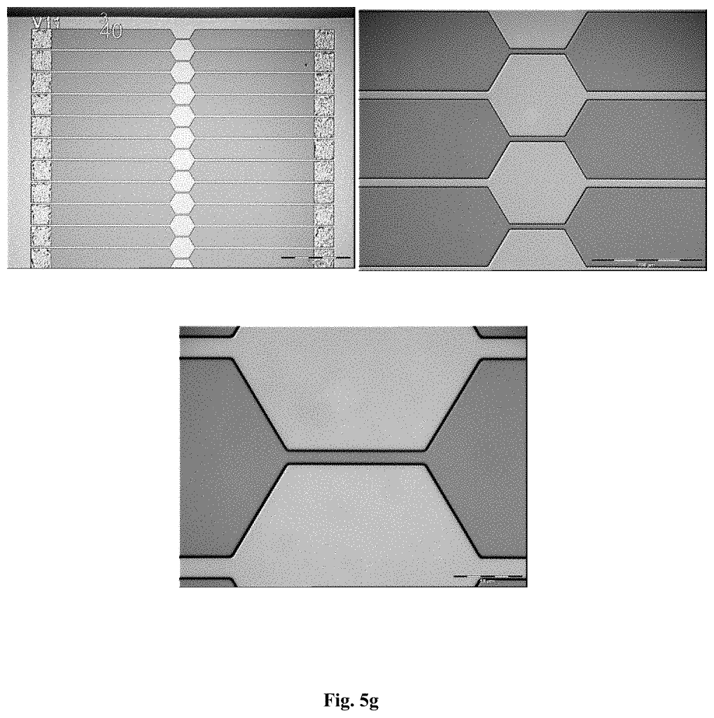

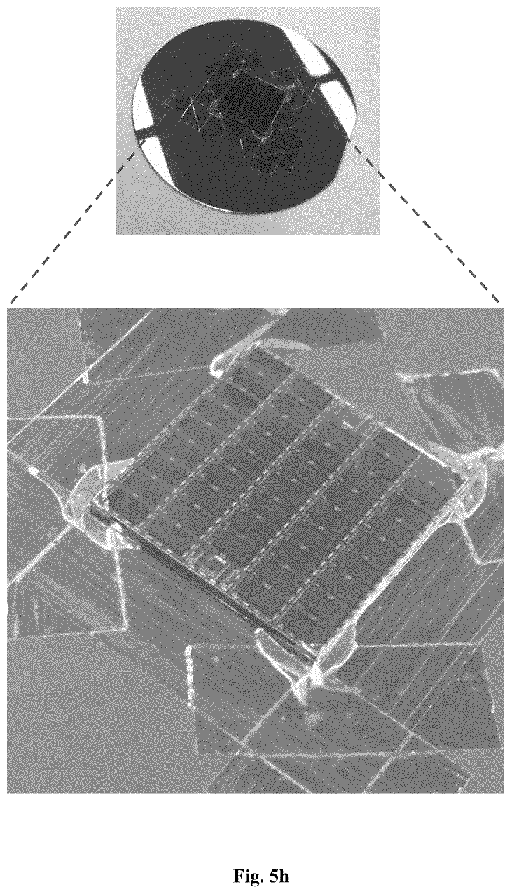

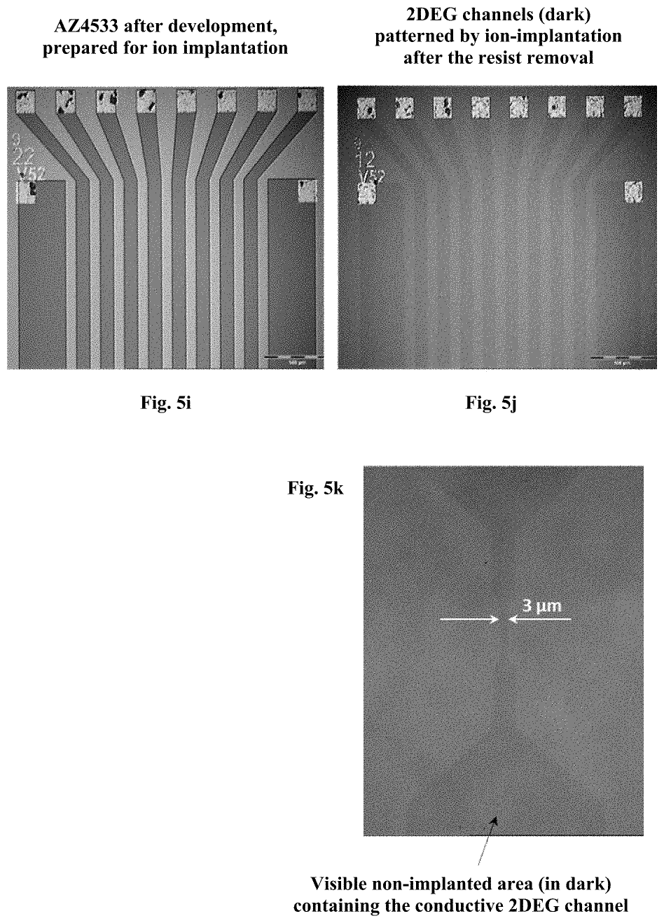

Reference is now made to FIGS. 5a-5c showing the sensor, which is obtained in Step 4 of the 2DEG-channel pattering. The lithography of the sensor was performed with AZ 4533, which is a positive thick resist having optimised adhesion for common wet etching. The lithographic resist film thickness obtained at 7000-rpm spin speed and at 100° C. for 1 min was 3 μm. Thus, as seen in the lithographic image of FIG. 5c, the formed 2DEG channel (13) is approximately 2-3 um wide. The overall exposure time was 9 sec, followed by 5-min development in MIF726 developer.

FIG. 5d-5e show the mask and corresponding lithographic image, respectively, of the sensor layout of the present invention. FIG. 5f demonstrates the high alignment precision of ±2-μm on 25×25 mm² samples in the lithography of the sensor layout of the present invention. FIG. 5g shows the lithographic images of the multichannel samples. FIG. 5h shows the fixed sensor chip sample on the Si—GaN/AlGaN wafer, which contains approximately 30-32 sensors with 4-8 channels on each sample and prepared for ion implantation. FIG. 5i shows the obtained lithographic image of the present sensor layout with the AZ4533 resist after development, prepared for ion implantation. FIG. 5j shows the 2DEG channels (dark) patterned by ion-implantation after the resist removal. The argon-ion implantation was conducted with 20 keV and 30 keV energies and with an exemplary dose of 2.5e¹³/cm² and a 7° tilt angle. AZ4533 was removed with oxygen plasma at 220 W for 10 min. FIG. 5k shows the visible non-implanted area containing the conductive 2DEG channel.

The atomic layer etching (ALE) performed in Step 8 of the manufacturing process is the most important stage in the process. As mentioned above, it allows the controlled recess of a top layer, removing a single atomic layer-by-layer, where the etch thickness is in the order of magnitude of a single atomic monolayer. As explained above, such ultra-low damage to the top layer of the heterogeneous structure, when the actual surface roughness is controlled by a single atomic monolayer, allows to achieve the sub-nanometre roughness (about 0.2 nm and less) of the top layer when its thickness is only few nanometres (5-9 nm).

The ALE process sequence consists of repeated cycling of process conditions. The total amount of material removed is determined by the number of repeated cycles. Each cycle is typically comprised of four steps: adsorption, first purge, desorption and second purge. During the adsorption step of the cycle, reactive species are generated in the reactor (for example, upon plasma excitation), adsorbed by, and react with material on the wafer. Due to the self-limiting process, and with the proper choice of reactants and process conditions, reaction takes place with only a thin layer of material, and the reaction by-products are formed. This step is followed by purging of the reactor to remove all traces of the reactant. Then the by-product desorption takes place due to bombardment of the wafer surface by noble gas ions with a tightly controlled energy. Again, by-products are purged from the reactor, and the wafer is ready for the last two (optional) steps of the manufacturing process.

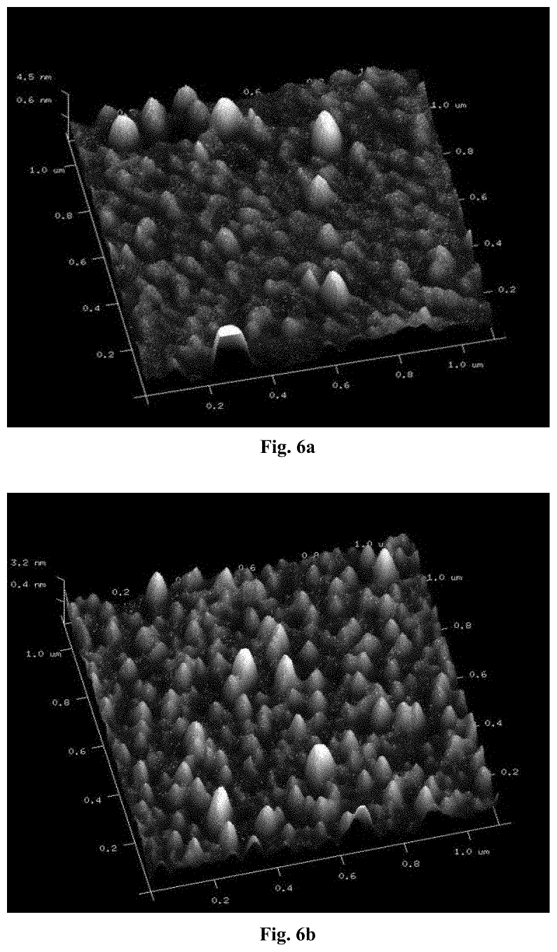

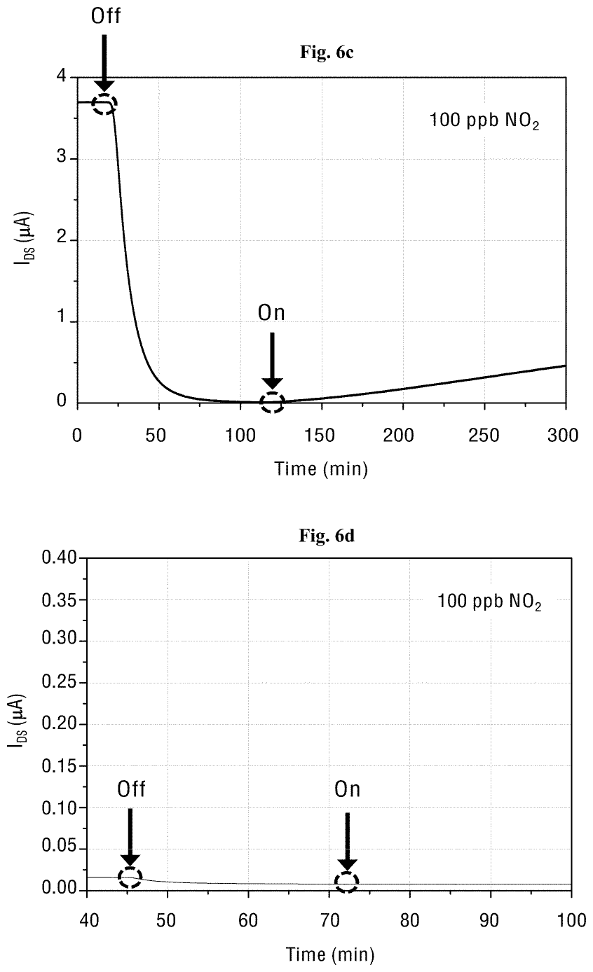

Reference is now made to FIG. 6a showing the AFM image of the top recessed layer surface of the PC-HEMT produced by the manufacturing process of the present invention. The measured RMS value of the surface roughness is 0.674 nm in this case. FIG. 6b shows the AFM surface image of the top recessed layer of the HEMT made by a conventional manufacturing process. In this conventional process, the HEMT initially had a top ultrathin-grown AlGaN layer of the 6-7 nm thickness. This layer was recessed with inductively-coupled plasma (ICP) for 60 sec using a conventional reactive-ion etching (ME) technique. The measured RMS value of the surface roughness is 1.211 nm in this case. FIGS. 6c show the time-dependent plot of the drain-source electric current I_{DS} of the nitrogen oxide sensor measuring 100 ppb of the NO₂ gas in 80%-humid air, where the sensor incorporates the PC-HEMT made by the manufacturing process of the present invention. FIGS. 6d show the time-dependent plot of the I_{DS} of the nitrogen oxide sensor measuring 100 ppb of the NO₂ gas in 80%-humid air, where the sensor incorporates and based on the HEMT made by the conventional manufacturing process. It is clear from these comparative examples that the manufacturing process of the present invention based on the ultra-low damaging ME with a narrow plasma-ion energy distribution leads to much lower roughness of the semiconductor surface, which in turn leads to incredibly high sensitivity of the sensor.

In a further aspect, the hetero-junction structure may be a three-layer structure consisting of two GaN layers and one AlGaN layer squeezed between said buffer layers like in a sandwich, wherein the top layer is a buffer layer. This may lead to formation of the two-dimensional hole gas (2DHG) in the top GaN layer above the AlGaN layer which results in reversing polarity of the transistor compared to the two-layer structure discussed above.

In general, polarity of III-V nitride semiconductor materials strongly affects the performance of the transistors based on these semiconductors. The quality of the wurtzite GaN materials can be varied by their polarity, because both the incorporation of impurities and the formation of defects are related to the growth mechanism, which in turn depends on surface polarity. The occurrence of the 2DEG/2DHG and the optical properties of the hetero-junction structures of nitride-based materials are influenced by the internal field effects caused by spontaneous and piezo-electric polarizations. Devices in all of the III-V nitride materials are fabricated on polar {0001} surfaces. Consequently, their characteristics depend on whether the GaN layers exhibit Ga-face positive polarity or N-face negative polarity. In other words, as a result of the wurtzite GaN materials polarity, any GaN layer has two surfaces with different polarities, a Ga-polar surface and an N-polar surface. A Ga-polar surface is defined herein as a surface terminating on a layer of Ga atoms, each of which has one unoccupied bond normal to the surface. Each surface Ga atom is bonded to three N atoms in the direction away from the surface. In contrast, an N-polar surface is defined as a surface terminating on a layer of N atoms, each of which has one unoccupied bond normal to the surface. Each surface N atom is also bonded to three Ga atoms in the direction away from the surface. Thus, the N-face polarity structures have the reverse polarity to the Ga-face polarity structures.

As described above for the two-layer heterojunction structure, the barrier layer is always placed on top of the buffer layer. The layer which is therefore recessed in the two-layer heterojunction structure is the barrier layer, specifically the AlGaN layer. As a result, since the 2DEG is used as the conducting channel and this conducting channel is located slightly below the barrier layer (in a thicker region of the GaN buffer layer), the hetero-junction structure is grown along the {0001}-direction or, in other words, with the Ga-face polarity. However, as explained above, the physical mechanism that leads to the formation of the 2DEG is a polarisation discontinuity at the AlGaN/GaN interface, reflected by the formation of the polarisation-induced fixed interface charges that attract free carriers to form a two-dimensional carrier gas. It is a positive polarisation charge at the AlGaN/GaN interface that attracts electrons to form 2DEG in the GaN layer slightly below this interface.

As noted above, polarity of the interface charges depends on the crystal lattice orientation of the hetero-junction structure, i.e. Ga-face versus N-face polarity, and the position of the respective AlGaN/GaN interface in the hetero-junction structure (above or below the interface). Therefore, different types of the accumulated carriers can be present in the hetero-junction structure of the embodiments.

In case of the three-layer hetero-junction structure, there are four possible configurations:

### Ga-Face Polarity

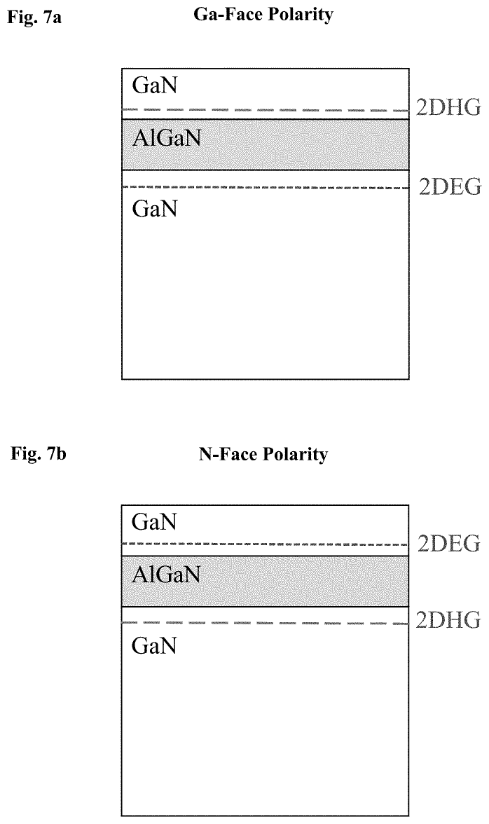

1) The Ga-face polarity is characterised by the 2DEG formation in the GaN layer below the AlGaN barrier layer. This is actually the same two-layer configuration as described above, but with addition of the top GaN layer. In this configuration, the AlGaN barrier layer and two GaN layers must be nominally undoped or n-type doped.
2) In another Ga-face configuration shown in FIG. 7a, in order to form the conducting channel comprising a two-dimensional hole gas (2DHG) in the top GaN layer above the AlGaN barrier layer in the configuration, the AlGaN barrier layer should be p-type doped (for example, with Mg or Be as an acceptor) and the GaN buffer layer should be also p-type doped with Mg, Be or intrinsic.

### N-Face Polarity

3) The N-face polarity is characterised by the 2DEG formation in the top GaN layer above the AlGaN barrier layer, as shown in FIG. 7b. In this case, the AlGaN barrier layer and two GaN buffer layers must be nominally undoped or n-type doped.
4) The last configuration assumes that the 2DHG conducting channel is formed in the buffer GaN layer below the AlGaN barrier layer. The top GaN layer may be present (three-layer structure) or not (two-layer structure) in this case. The AlGaN barrier layer must be p-type doped (for example, with Mg or Be as an acceptor) and the bottom GaN layer should be also p-type doped with Mg, Be or intrinsic.

Thus, there are four hetero-junction three-layer structures implemented in the transistor of the embodiments, based on the above configurations:

A. Ga-Face GaN/AlGaN/GaN heterostructure with the 2DEG formed in the GaN buffer layer below the AlGaN barrier layer. In this case, the top GaN layer may be omitted to obtain the two-layer structure. For the three-layer structure, the top GaN layer must be recessed to 1-9 nm thickness in the open gate area or grown with this low thickness, with the roughness below 0.2 nm, and the thickness of the AlGaN barrier can be adjusted properly during growth
B. Ga-Face GaN/AlGaN/GaN heterostructure with the 2DHG conducting channel formed in the top GaN layer above the AlGaN barrier layer. The top GaN layer must be recessed to 5-9 nm thickness in the open gate area with the roughness below 0.2 nm, and the thickness of the AlGaN barrier layer can be adjusted properly. P-type doping concentrations of the GaN layer and AlGaN barrier have to be adjusted; the 2DHG has to be contacted (in the ideal case by ohmic contacts).
C. N-Face GaN/AlGaN/GaN heterostructure with the 2DEG in the top GaN layer above the

AlGaN barrier layer. The top GaN layer must be recessed to 5-9 nm thickness in the open gate area with the roughness below 0.2 nm. Thickness of the AlGaN barrier can be adjusted during growth. N-type doping levels of the GaN buffer layer and the AlGaN barrier layer must be adjusted; the 2DEG has to be contacted (in the ideal case by ohmic contacts).

D. N-Face GaN/AlGaN/GaN heterostructure with the 2DHG in the GaN buffer layer below the AlGaN barrier layer. In this case, the top GaN layer may be omitted to obtain the two-layer structure. In both, the two-layer and three-layer configurations, the top GaN layer must be recessed to 1-9 nm thickness in the open gate area with the roughness below 0.2 nm, and the thickness of the AlGaN barrier can be adjusted properly.

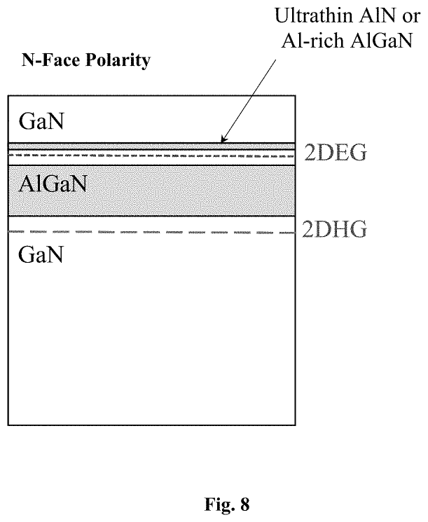

In all the above structures, the deposition of a dielectric layer on top might be beneficial or even necessary to obtain a better confinement (as in case of the N-face structures). As shown in FIG. 8, for the above “C” structure, it may be even more beneficial to include an ultrathin (about 1 nm) AlN or AlGaN barrier layer with high Al-content on top of the 2DEG channel to improve the confinement.

The preferable structures of the embodiments are structures “B”, “C” and “D”. In the structure “B”, the 2DHG conducting channel is formed in the top GaN layer, which has a higher chemical stability (particularly towards surface oxidation) than the AlGaN layer. Concerning the structure “C”, the 2DEG conducting channel might be closer to the surface. Therefore, the electron mobility might be lower than in the 2DEG structure with the Ga-face polarity. In general, the polarity of the heterostructure can be adjusted by the choice of the substrate (e.g. C-face SiC) or by the growth conditions.

Based on the above, one of the aspects of the present application is an open-gate pseudo-conductive high-electron mobility transistor (PC-HEMT) for amplifying signals in a sub-THz and THz frequency range, comprising:

(1) a multilayer hetero-junction structure made of gallium nitride (GaN) and aluminium gallium nitride (AlGaN) single-crystalline or polycrystalline semi-conductor materials, deposited on a substrate layer, and characterised in that:
(a) said structure comprises (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer;
(b) said structure comprises (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
(c) said structure comprises (i) one top AlGaN layer recessed in an open gate area of the transistor to the thickness of 5-9 nm and having the surface roughness of 0.2 nm or less, and
(ii) one bottom GaN buffer layer; said layers have N-face polarity, thus forming a two-dimensional hole gas (2DHG) conducting channel in the GaN buffer layer, close to the interface with said AlGaN barrier layer;
(2) source and drain contacts connected to said 2DEG or 2DHG conducting channel and to electrical metallisations for connecting said transistor to an electric circuit; and
(3) a Vivaldi antenna electrode placed on a top (GaN or AlGaN) layer between said source and drain contact areas in an open gate area of the transistor and capable of detecting electrical signals in the sub-THz- and THz-frequency ranges of 30 GHz to 300 THz (the wavelength range is about 1 cm to 1 μm).

Another important feature of the sensor of the present application is that an electrical connection of the heterojunction structure to the 2DEG or 2DHG channel is realised via capacitive coupling to the electrical metallisations through a Schottky barrier contact. “Capacitive coupling” is defined as an energy transfer within the same electric circuit or between different electric circuits by means of displacement currents induced by existing electric fields between circuit/s nodes. In general, ohmic contacts are the contacts that follow Ohm's law, meaning that the current flowing through them is directly proportional to the voltage. Non-ohmic contacts however do not follow the same linear relationship of the Ohm's law. In other words, electric current passing through non-ohmic contacts is not linearly proportional to voltage. Instead, it gives a steep curve with an increasing gradient, since the resistance in that case increases as the electric current increases, resulting in increase of the voltage across non-ohmic contacts. This is because electrons carry more energy, and when they collide with atoms in the conducting channel, they transfer more energy creating new high-energy vibrational states, thereby increasing resistance and temperature.

When electrical metallisations are placed over single-crystalline or poly-crystalline semiconductor material, the “Schottky contact” or “Schottky barrier contact” between the metal and the semiconductor occurs. Energy of this contact is covered by the Schottky-Mott rule predicting the energy barrier between a metal and a semiconductor to be proportional to the difference of the metal-vacuum work function and semiconductor-vacuum electron affinity. However, this is an ideal theoretical behaviour, while in reality most interfaces between a metal and a semiconductor follow this rule only to some degree. The boundary of a semiconductor crystal abrupt by a metal creates new electron states within its band gap. These new electron states induced by a metal and their occupation push the centre of the band gap to the Fermi level. This phenomenon of shifting the centre of the band gap to the Fermi level as a result of a metal-semiconductor contact is defined as “Fermi level pinning”, which differs from one semiconductor to another. If the Fermi level is energetically far from the band edge, the Schottky contact would preferably be formed. However, if the Fermi level is close to the band edge, an ohmic contact would preferably be formed. The Schottky barrier contact is a rectifying non-ohmic contact, which in reality is almost independent of the semi-conductor or metal work functions.

Thus, a non-ohmic contact allows electric current to flow only in one direction with a non-linear current-voltage curve that looks like that of a diode. On the contrary, an ohmic contact allows electric current to flow in both directions roughly equally within normal device operation range, with an almost linear current-voltage relationship that comes close to that of a resistor (hence, “ohmic”).

Since the source and drain contacts are non-ohmic (i.e. capacitively-coupled), the DC readout cannot be carried out. To electrically contact the 2DEG/2DHG channel underneath, about 5-20 nm bellow the metallisations, the AC-frequency regime must be used. In other words, the AC readout or impedance measurements of the electric current flowing through the 2DEG/2DHG-channel should be performed in this particular case. The capacitive coupling of the non-ohmic metal contacts with the 2DEG/2DHG channel becomes possible only if sufficiently high AC frequency, higher than 30 kHz, is applied to the metallisations. To sum up, the electrical metallisations, which are capacitively coupled to the 2DEG/2DHG channel utilise the known phenomenon of energy transfer by displacement currents. These displacement currents are induced by existing electrical fields between the electrical metallisations and the 2DEG/2DHG conducting channel operated in the AC frequency mode through the Schottky contact as explained above.

In one embodiment, a tapered slot antenna, also known as a Vivaldi antenna is placed in the open gate area of the transistor. The Vivaldi-antenna gate is capable of detecting various sub-THz and THz-frequencies (in the frequency range of about 30 GHz to 300 THz). By applying additional plasmonic filters of any kind, the frequencies could be tuned precisely to a specific frequency of choice.

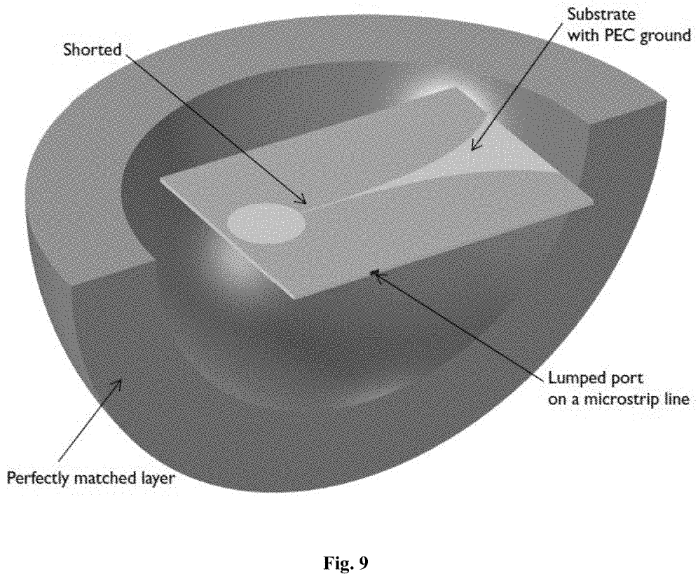

In general, a Vivaldi antenna is a co-planar broadband-antenna, which is made from a dielectric plate metalized on both sides. FIG. 9 shows the model of the Vivaldi antenna realised on a thin dielectric substrate. An exponential function is used for the taper profile. The entire domain is bounded by a perfectly matched layer. The objective of this model is to compute the far-field pattern and to compute the impedance of the structure. Good matching is observed over a wide frequency band. In this model of the Vivaldi antenna, the tapered slot is patterned with a perfect electric conductor (PEC) ground plane on the top of the Si dielectric substrate. One end of the slot is open to air and the other end is finished with a circular slot.

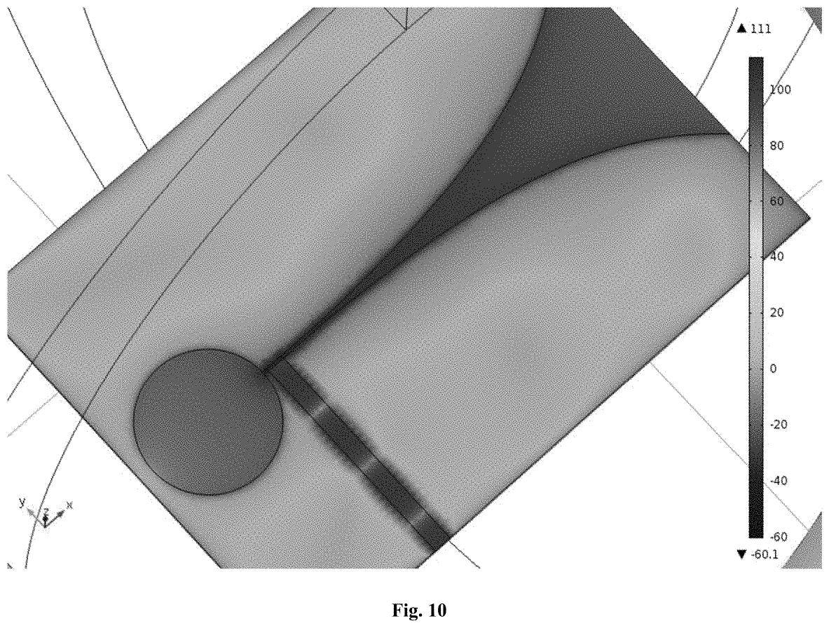

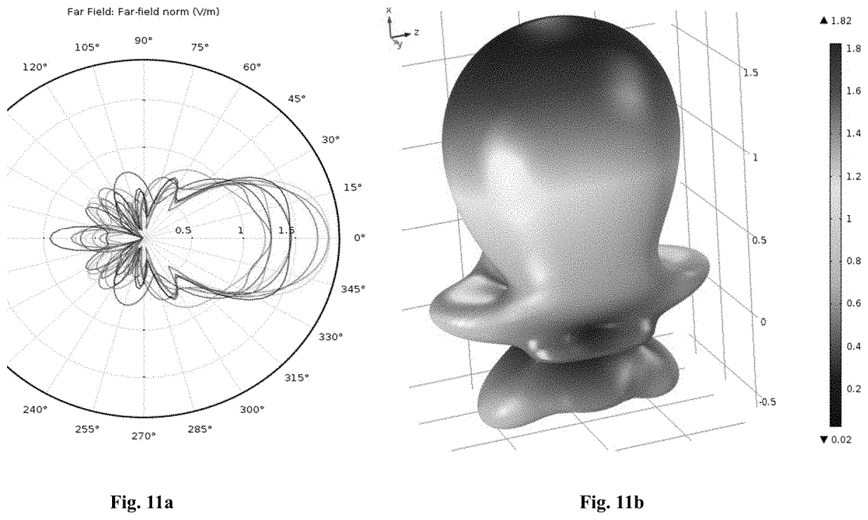

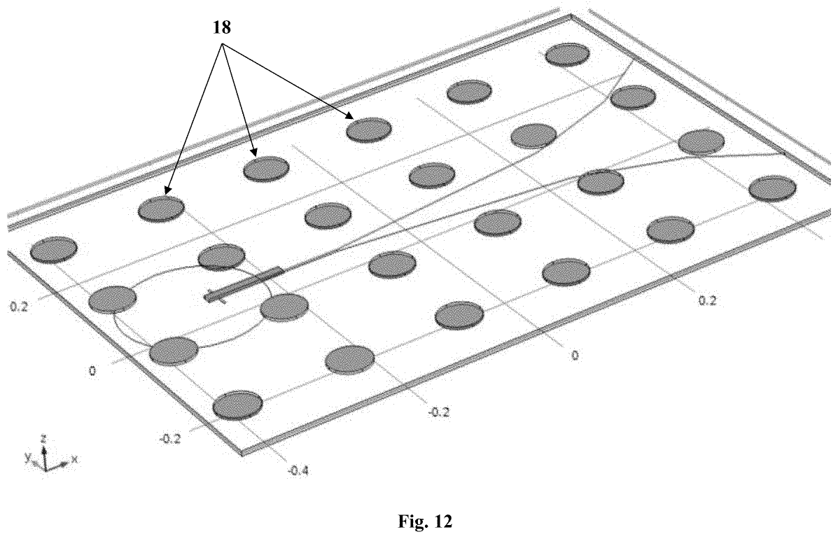

On the bottom of the substrate, the shortened 50 Ohm microstrip feed line is modelled as the PEC surfaces. The entire modelling domain is bounded by a perfectly matched layer (PML) chamber absorbing all radiated energy. To excite the antenna, a lumped port is used. FIG. 10 shows the electric field distribution in the Vivaldi antenna plane at 480 GHz. The substrate size is 917×667×4 μm³ with total area about 0.6 mm². Substrate material is a high-dielectric constant, low conductive semiconductor material with σ>1000 Ohm·cm, such as for example, Si and GaAs. The following table provides the exemplary sub-THz Vivaldi antenna characteristics:

| Parameter | Units | Value | Comments |
|---|---|---|---|
| Substrate size | μm³ | 917 × 667 × 4 | Si, GaAs (high |
| | | | dielectric constant |
| | | | material) |
| Metal layer thickness | nm | 80 | Au |

FIG. 11a shows the far-field directional radiation pattern of the Vivaldi antenna starting from 240 GHz till 780 GHz and FIG. 11b shows the corresponding 3D far-field pattern at 480 GHz, with the maximum in the X direction (axis along the substrate), which means this is a unidirectional antenna. As seen in FIGS. 10 and 11a-11b, the feeding line excites a circular space via a microstrip line, terminated with a sector-shaped area. From the circular resonant area the energy reaches an exponential pattern via a symmetrical slot line. In addition, the frequency response SWR of the Vivaldi antenna shows wide-band impedance matching better than 2:1 in most of the simulated frequency range. Printed circuit technology makes this type of antenna cost effective at sub-THz and also THz frequencies.

Advantages of the Vivaldi antenna is its broadband characteristics suitable for ultra-wideband signals in the sub-THz and THz frequency domain, its relatively easy manufacturing process using common methods for PCB production, and its easy impedance matching to the feeding line using microstrip line modelling methods. Also, the Vivaldi antenna has been chosen because it permits to integrate a long meander delay without having undesired effects.

The Vivaldi antenna is a reciprocal device. It collects in a passive mode exactly the same frequencies that can be actively radiated. Due to its very broadband character, the Vivaldi antenna may receive signals outside the 240 GHz-780 GHz range. In order to limit the antenna sensitivity to the desired 0.2 THz-0.8 THz range, two-dimensional photonic crystals (18) may be superimposed on top of the antenna metal layer, as shown in FIG. 12. The exemplary dimensions of such Vivaldi antenna and lattice constant of the photonic crystal are summarised in the following table:

| Parameter | Units | Value | Comments |
|---|---|---|---|
| Antenna substrate | μm | 4 | Si, GaAs (high dielectric |
| | | | constant material) |
| Device footprint area | μm² | 917 × 667 | |
| Metal layer thickness | nm | 80 | Au |
| Superimposed Si thickness | μm | 4 | Si or GaAs |
| Distance between the pillars | μm | 160 | Au or Al |
| Pillar diameter | μm | 60 | |
| Pillar height | μm | 4 | |

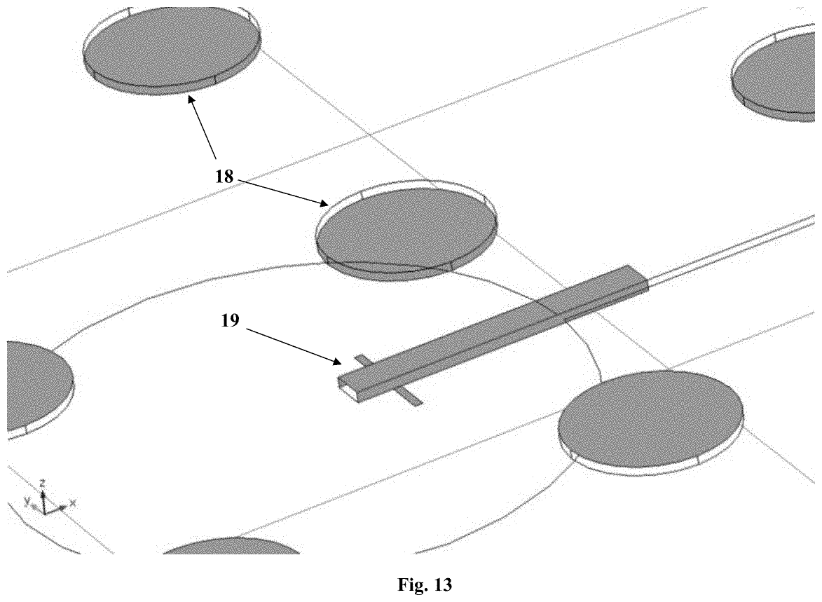

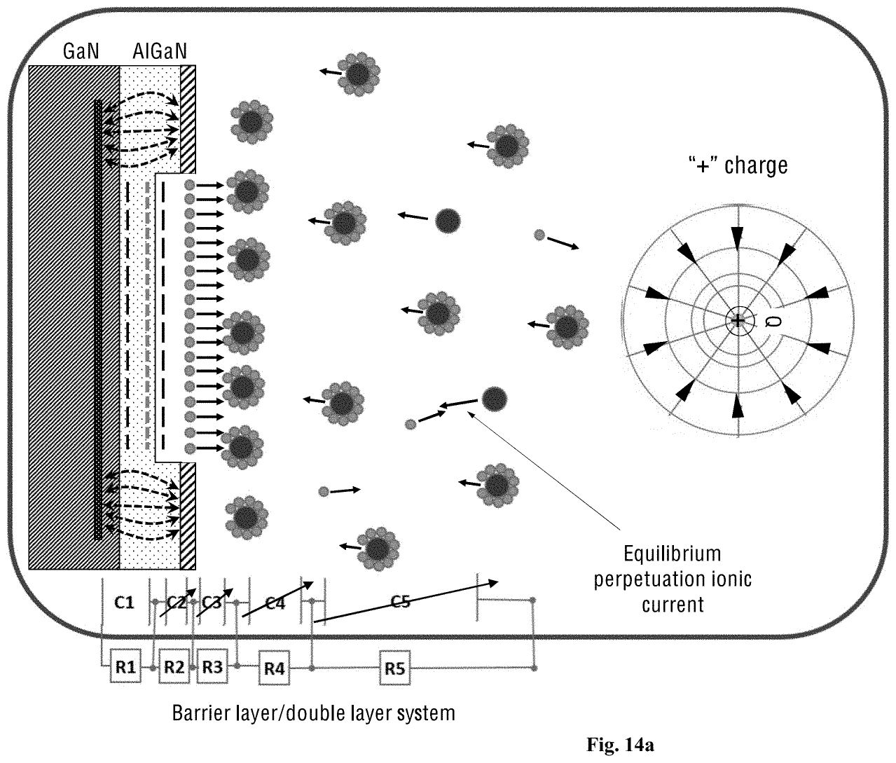

FIG. 13 shows the position of the metal connector (19) on the Vivaldi antenna for coupling with the PC-HEMT of the embodiment.

In another embodiment, the transistor of the present invention further comprises at least one molecular or biomolecular layer (20) immobilised on the surface of the transistor within the open-gate area (17) for sensing target chemical compounds or biomolecules (analytes). The molecular or biomolecular layer specific layers (20) allow for example, gas molecules to be bound or adsorbed and then detected. This molecular or biomolecular layer (20) may further increase sensitivity and selectivity of the sensor based on the transistor of the present invention. The (bio)molecular layer (20) is made, for example, of polymers, redox-active molecules, such as phthalocyanines, metalorganic frameworks, such as metal porphyrins, for example hemin, biomolecules, for example receptors, antibodies, DNA, aptamers or proteins, water molecules, for example forming a water vapour layer, such as a boundary surface water layer, oxides, semi-conductive layer or catalytic metallic layer. The molecular or biomolecular layer (20) is immobilised over either a portion of the transistor's top layer surface or substantially over its entire surface in the open-gate area to further improve sensitivity of the sensor for detection of target molecule or analyte.

In general, the molecular or biomolecular specific layer (20) is any coating that adsorbs selected chemicals present in the environment. The presence of molecules or biomolecules adsorbed on the layer (20) modulates the signal received by the transistor due to shifting equilibrium between the 2DEG/2DHG channel density and the number of ionised surface donors which is governed by charge neutrality and continuity of the electric field at interfaces. Measurement of this modulation is used to indicate the identity and concentration of specific molecules or biomolecules in the environment.

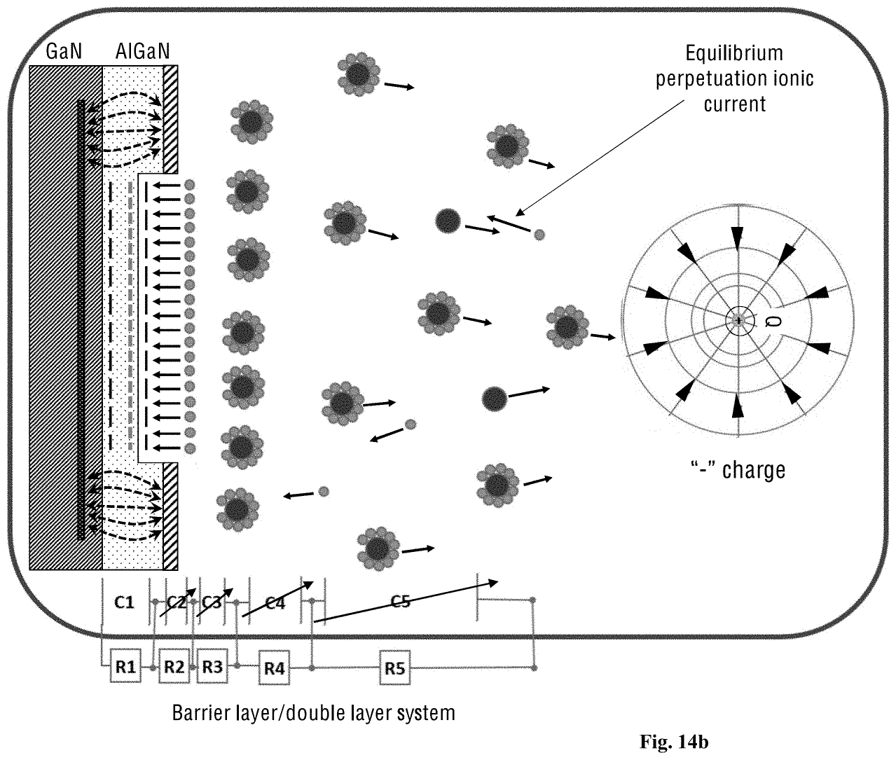

Reference is now made to FIGS. 14a-14b illustrating the barrier layer/liquid or gas interface with the double layer formation, simplified equivalent interface circuity and ion electrodynamics during exposure of the sensor to a positive charge (FIG. 14a) and a negative charge (FIG. 14b). When immersed into a gas or liquid environment, any surface potential causes natural formation of an electrochemical double layer at the contact interface to maintain charge equilibrium between the solid state and ionic conductive liquid or gas.

In FIGS. 14a-14b, this double layer is shown together with the simplified equivalent circuitry at the interface. The double layer is created with a 1- to 3-nm-thick sharp separation between the negative and positive ion space charge zones C2-R2 and C3-R3, which cause a secondary space charge equilibrium zone C4-R4 (10 nm to 1 μm) and charge gradient zone C5-R5 disappearing in the bulk liquid or gas. When there is no more potential shift from the solid and from the liquid or gas, then the charge equilibrium is maintained with C1/R1-C5/R5 elements possessing a quasi-constant values.

Ion flow is schematically shown in FIGS. 14a and 14b with vector arrows during an electrodynamic rearrangement when an external charge is introduced into an equilibrated electrolyte. FIG. 14a shows the electrodynamic rearrangement with an external positive charge, and FIG. 14b shows the electrodynamic rearrangement but with an external negative charge. When the ions react to an external electric field applied in the liquid, the equivalent circuitry mirroring the space charges changes accordingly. Since the PC-HEMT of the present application is extremely sensitive to any smallest surface charge changes (C1/R1) due to its pseudo-conductivity, as explained above, rearrangement of the gradient ions in the shown space charge zones from C5/R5 to C2/R2 is capable of modulating the 2DEG conductivity. Dynamics and magnitude of the newly formed equilibrium at each time moment is directly proportional to the liquid electrolyte conductivity, ions mobility and external charge value, therefore defining the resulting electrolyte charge. In general, any electrolyte strongly enhances the sensor charge response due to the excellent direct charge transfer towards the barrier layer/electrolyte interface. The ions of the liquid or gas interact directly with the super sensitive surface trap states of the ultrathin barrier layer.

The above phenomenon occurring at the PC-HEMT surface, discovered by the present inventors, is defined as an “intra-fluid ionic interaction”. Thus, if this transistor connected to a circuit is immersed into an ion conductive fluid (being liquid or gas), then ions of the fluid start electro-dynamically react to any external charge by their movement. Being in direct contact to the barrier layer surface, the charge sensitivity is tremendously enhanced. The fluid acts in this case as an additional antenna (additional to Vivaldi antenna) perfectly matching the 2DEG transducer. Electric charges generated in any environment, as well as their super position dipole, are projected to this fluid antenna, in which the transistor is immersed.

To sum-up, in a direct current (DC) mode, the top layer-to-fluid-interface of the PC-HEMT is in charge equilibrium, where the 2DEG is directly incorporated as a balancing polarisation element. Once an external electric charge (originated from dipole molecules forming a layer or two neutral molecules creating a dipole pair under London forces) is introduced into the electrolyte fluid environment, the net charge equilibrium is shifted resulting in a change in the electron density and mobility. In case of the pseudo-conducting 2DEG channel, it becomes easily modulated and the strongest amplification phenomenon is observed. Sensitivity of the PC-HEMT in this case is so ultra-high that it allows detecting neutral molecules diffusing to the surface and coupling to the PC-HEMT top layer surface via a getter effect changing the surface trap states. The getter effect actually allows the sensor based on the PC-HEMT of the present invention to collect free gases by adsorption, absorption or occlusion.

In radiofrequency (RF) mode, when the electric current in the pseudo-conducting 2DEG channel is alternating (AC), the near-field and displacement current coupling effects at electrochemical double layers take place. In that case, the super-Debye interactions allow detection of any ion types selectively at MHz frequency range and ion solvation shells and resonance frequencies of intra-fluid ion-ion interaction at GHz range.

As discussed above, at any solid state/electrolyte interface, the capacitive and resistive elements of the sensor form an electrochemical surface potential originated from an interaction between the surface trap states and a double layer capacity, while the interaction between the 2DEG and the surface trap states originates from tunnelling and electrostatics. It has now been surprisingly found that operation of the PC-HEMT sensor as an open gate field-effect transistor is not required in order to modulate the surface electrochemical potential within the barrier layer/electrolyte system.

It has recently been found by the present inventors that the PC-HEMT of the invention is capable of overcoming the Debye length limitation. The overall design of the transistor enables its additional operation in the frequency domain and helps to stabilise the electronic readout when recording very small DC current changes. Therefore, the sensors of the invention based on the PC-HEMT can be used in impedance spectroscopy applications. A combination of potentiometric and impedimetric readout enables a more reliable sensing of molecules with the potential to sense beyond the Debye screening of electrical charges in an electrolyte solution, which is usually the limiting factor in most of the sensors having only potentiometric or conductometric readout.

As mentioned above, the PC-HEMT of the present invention is functionalised optionally with different molecules (receptors), which are capable of binding to a target (analyte) molecule, for sensing. As a result, the PC-HEMT-based sensor of the invention can be used for label-free detection of target (analyte) molecules by monitoring changes in the electric current of the transistor caused by variations in the charge density or the impedance at the open gate-electrolyte interface.

In general, charges formed in a liquid medium sensed by ISFETs come from the dissolved molecules. Depending on the pH value of the liquid and the molecules' isoelectric point, the dissolved molecules exhibit a global charge. However, this charge may be non-uniformly distributed over the molecule. In addition, the molecules have different sizes and a different 3D structure. Therefore, it is very important that:

the sensor's interface is chemically engineered in a very uniform and reproducible manner,
receptors need to be immobilised on the sensor's surface as highly selective receptor layer with a very uniform grafting density,
the sensor should have redundant structure exhibiting multiple sensors cancelling out wrongly functionalised transistors, and
a molecular friendly surface architecture and microenvironment with fixed pH value, fixed ionic strength and temperature needs to be established to avoid denaturation of the molecules on the sensor surface.
The latter is controlled by respective reference sensors for temperature, pH value and ionic strength in the sensor chip design. However, even with the above-mentioned ideal sensor design it can be the case that the potentiometric detection of charges, which lead to changes in surface potential and hence, to a shift of the ISFET threshold voltage, cannot be detected, because the relevant charges are located outside the Debye screening length of the liquid electrolyte.

In most cases (in most biosensors), molecular receptors bound to the transistor surface are spatially separated from this surface by molecular cross-linkers or proteins of approximately 5-15 nm length. Therefore, the aforementioned charges are screened from the sensing surface by dissolved counter ions. As a result of the screening, the electro-static potential that arises from charges on the analyte molecule exponentially decreases to zero with increasing the distance from the sensing surface. This screening distance is defined as a “Debye length”, and it must be carefully selected when designing the receptor layer of any ISFET in order to ensure the optimal sensing. For example, when detecting molecules in blood serum, the typical Debye screening length is 0.78 nm at temperature 36° C. with an electrical permittivity of 74.5 for water. This means that after this length, the Debye screening length is given by:

$${λ}_{D} = \sqrt{\frac{{ϵ}_{r} {ϵ}_{0} {k}_{b} T}{2 {n}_{0} {z}^{2} {e}^{2}}}$$

where n₀ is the bulk concentration of the electrolyte, ϵᵣ is the relative dielectric permittivity of the solvent (in case of water at 36° C. a value of 74.5), ϵ₀ is the permittivity of the vacuum, k_{b} is the Boltzmann constant, T is the temperature, z is the ion charge, and e is the elementary charge.

The screening length means that an electrical field originating from a point charge is dropped to its 1/e value (29%) in this length. Because of this limitation, charges from larger biomolecules (5-15 nm) cannot be detected in a serum sample. To overcome this problem, the charges should be attracted closer to the sensor surface by using very short length receptors or by operating the sensor in completely desalted buffers for electronic molecular detection.

Thus, the Debye length limitation can be overcome by modification of the receptors and controlling the immobilisation density over the ISFET's sensing surface. Roey Elnathan et al 2012 (in “Biorecognition Layer Engineering: Overcoming Screening Limitations of Nanowire-Based FET Devices”, Nanoletters 12, 2012, pp. 5245-5254) described this approach in detail and demonstrated the increased sensitivity of their sensor to troponin detection directly from serum for the diagnosis of acute myocardial infarction. However, the present inventors made a step forward and proposed to sense beyond the Debye screening length without modification of the receptors, but operating the PC-HEMT of the present invention at high frequencies and using a combined transducer principle, which is one of the aspects of the present invention. It will be described below in details. By combination of a precise monitoring and control of the main parameters, temperature, pH and ionic strength with an array of electronically identical PC-HEMTs of the present invention, and a highly reproducible and uniform bio-receptor surface layer, the precise identification of biomolecules can be obtained.

By using coatings with selective adsorption properties, sensors detecting specific chemical or biological compounds for both gas-phase and liquid-phase environments have been now developed by the present applicants. Typically, durable oxide-based coatings that are chemically modified to provide the required adsorption characteristics are used. These coatings can selectively adsorb ionic species from solution for use in applications such as monitoring electroplating processes or waste streams for toxic metals such as chromium, cadmium, or lead.

Polymer coatings that adsorb a wide variety of chemicals are ideally suited for monitoring the highly regulated ozone-depleting chlorinated hydrocarbons. Simultaneous measurement of the wave velocity and attenuation can be used to identify chemical compounds and their concentration. One of the applications of the sensors of the present invention is the selective detection of organophosphates, which are a common class of chemical warfare agent. The detection of these chemicals is done by the active chemical layer (20) composed of thin films of self-assembled monolayers. The sensitivity of these films on the piezoelectric material of the sensor endows the sensor with immunity to interference from water vapour and common organic solvents while providing sensitivity in the part-per-billion concentration of organophosphates. As a result, arrays of such sensors with appropriate coatings can be used to detect the production of chemical weapons.

Another application of the sensors of the present invention is a chemical detection and analysis of environmentally toxic compounds and toxins, such as food toxins, for example aflatoxin, neurotoxic compounds, for example lead, methanol, manganese glutamate, nitrix oxide, Botox, tetanus toxin or tetrodotoxin, shellfish poisoning toxins, for example saxitoxin or microcystin, Bisphenol A, oxybenzone and butylated hydroxyanisole. In general, chemical detection and analysis of toxic compounds can be aimed at determining the level or activity of these compounds in the emission sample (into which the toxic compound is incorporated en route to human exposure, for example in industrial effluents), in the transport medium (for example, air, waste water, soil, skin, blood or urine), and at the point of human exposure, for example in potable water. Sensing the emission sample, the transport medium, and the point of human exposure may be necessary for a comprehensive plan designed both to detect toxic compounds, analyse them and to exert control on the emission of the toxic compounds in order to achieve hazard reduction.

For a given toxic analyte, chemical sensors of the present application will certainly differ in sensitivity, selectivity, or other characteristics, which may be required to monitor the emission sample, the transport medium, and individual exposure. Concentration of a toxic compound is typically greater in the emission sample than after dispersal in a transport medium and can vary widely. The physical and chemical properties of the analyte and its immediate environment (airborne vapour, contained in solid or liquid aerosol, chemically or photochemically reactive and decomposing into compounds of different toxicity, radioactive, ionic, acidic or lipophilic) are also influential in the design of a suitable configuration for the sensor of an embodiment.

Still another application of the sensors of the present invention is a chemical detection of explosives. In general, a large range of explosives can be detected with the sensor of an embodiment. A distinction is made between the bulk explosives and the trace explosives. In case of the trace explosives, the sensor is capable of detecting vapours of the explosive chemicals, thereby detecting the trace quantities emitted from explosive materials either directly in the environment or in the particulates of explosive materials that have been collected and then vaporised in the laboratory within the analytical instrument. The sensor of an embodiment can be operated both by direct sampling of the air containing the trace explosive vapours as well as by vaporising a sample that was collected by swiping a surface contaminated with explosive particulates.

Apart from simply being able to detect explosive materials, the sensor of the present invention is capable of identifying and quantifying the explosives. In general, a sensor that is used as a safety measure at airports will have other requirements than one that will be used in the field during military missions. Therefore, the configuration of the sensor can vary dependent on the particular application. There are different requirements to the throughput and, because of elevated background levels in military environments, the dynamic range. Furthermore, the military sensor for detection and analysis of explosives should be portable compared to the fixed sensors in laboratories or airports. Another consideration is the difference between detection and identification. In some instances a device will be used to sense whether a certain explosive material is present, whereas in others it is also necessary to determine which explosive compound it is. Furthermore, it can be important to consider how many different compounds, or groups of compounds, one device must be able to detect or identify. Different sensor configurations described below meet the above requirements for different types of the sensors.

Instead of detecting the explosive compounds themselves, the sensors of the present invention may also be used to detect other materials that could indicate the presence of an explosive material. These “other” materials are actually associated compounds that tend to be present when explosives are present, such as decomposition gases or even taggants, materials that have been added during the production of the explosive to facilitate the detection. An advantage of this approach is that taggants and some associated compounds have a higher vapour pressure than the explosive compound itself, and are thus easier to detect. In addition to the sensitivity, the selectivity of the sensor should also be considered. The selectivity of the sensors of an embodiment to vapours of the trace explosives may be increased by using them in an array. By using the sensors in an array it is possible to obtain a signal similar to an artificial olfactory system of a nose when the responses of a number of sensors are combined to give a fingerprint-like signal. In this case, pattern recognition methods, such as multiple axes radar plots, can be used to analyse the signal, match it to known responses from a database, and thus identify the explosive.

Examples of the explosive materials detected by the sensors of the invention in aqueous medium are picrates, nitrates, trinitro derivatives, such as 2,4,6-trinitrotoluene (TNT), 1,3,5-trinitro-1,3,5-triazinane (RDX), N-methyl-N-(2,4,6-trinitrophenyl)nitramide (nitramine or tetryl), pentaerythritol tetranitrate (PETN), trinitroglycerine, nitric esters, derivates of chloric and perchloric acids, azides, and various other compounds that can produce an explosion, such as fulminates, acetylides, and nitrogen rich compounds such as tetrazene, octahydro-1,3,5,7-tetranitro-1,3,5,7-tetrazocine (HMX), peroxides (such as triacetone trioxide), C4 plastic explosives and ozonides. In addition to the explosives, nitrobenzene, 2, 4-dinitrotoluene and several other organic compounds were tested being concomitant chemicals of TNT or some common water pollutants. The biomolecular layer (20) can be for example, a layer of the antibodies immobilised against a specific explosive compound. Alternatively, the molecular layer (20) can be phthalocyanine system having 2,2,3,3-tetrafluoropropyloxy substituents or cyclodextrin as sensitive materials for the detection of different explosives in aqueous media, in particular nitro-containing organic compounds.

As mentioned above, a biomolecular layer (20) sensitive to a certain target biomolecule, such as a specific pathogen, may be deposited on the top layer surface of the PC-HEMT within the open-gate area (17). As a result, upon binding of the specific pathogen, not only an electric field change, but also a mass change is normally observed and the density of the target molecules can be detected and further correlated to its concentration. In a further embodiment, the PC-HEMT of the present invention is placed on free standing membranes and used in “pressure-sensitive” mode, thus being capable of measuring very small pressures. Such sensor uses the free-standing membranes for creating a mass-loading effect which makes it possible to increase selectivity of the sensors via adding mechanical stress (mass-loading effect) as an additional parameter of the PC-HEMT-based sensor. The free-standing membranes are extremely flexible free-standing columns of substrate composed of sapphire, silicon, silicon carbide, gallium nitride or aluminium nitride, preferably gallium nitride, having thickness of 0.5-2 μm. The free-standing substrate membranes are very sensitive to any tensile, compressive or mechanical stress changes on the surface of the multilayer hetero-junction structure. This results in a mass loading effect, which will be discussed below.

In general, mechanical sensors, much like pressure sensors, are based on the measurement of the externally induced strain in the heterostructures. The pyroelectric properties of group-III-nitrides, such as gallium nitride (GaN), allow two mechanisms for strain transduction: piezoelectric and piezoresistive. The direct piezoelectric effect is used for dynamical pressure sensing. For measurements of static pressure, such sensors are not suitable due to some leakage of electric charges under the constant conditions. For static operation, the piezoresistive transduction is more preferable.

Piezoresistive sensors using wide band gap materials have been previously employed using hexagonal silicon carbide bulk materials for high temperature operation. The GaN/AlGaN structures piezoresistivity is comparable to silicon carbide. However, piezoresistivity can be further amplified by HEMT structure. For piezoresistive strain sensing at relatively lower pressures (or pressure differences), diaphragm or membranes should be used, where the external pressure is transferred into a changed internal strain caused by bending. The resulting change in polarization alters the 2DEG channel current which is measured. Thus, the sensor configuration of the present invention may involve the piezoelectrically coupled, charge and mass sensitive, free-standing GaN membranes, which are prepared, for example, according to U.S. Pat. No. 8,313,968, and offer an elegant and effective solution to achieve both downscaling and an integrated all-electrical low-power sensing-actuation. As mentioned above, GaN exhibits both, piezo- and pyro-electrical properties, which can be functionally combined. Whereas the piezoelectricity enables realisation of an integrated coupling mechanism, the 2DEG additionally delivers a pronounced sensitivity to mechanical stress and charge, which allows the sensor to use the pyroelectric effects. The dynamic change in 2DEG conductivity is also caused by a change in piezoelectric polarisation.

In other words, the sensor of the present invention may also act as a miniature analytical balance, weighing the biological pathogens that bind to its surface. For example, biological pathogens may be captured very selectively by the biomolecular layer (20) consisting of specific biological receptor molecules, such as antibodies, short peptide chains or single-strand DNAs, which are capable of distinguishing between closely related pathogens. In fact, one can think of the sensor of an embodiment as a spring with a small weight bouncing at one end. As the molecule becomes attached to the sensor, the weight on the spring increases, which causes the speed of the spring's oscillation to significantly decrease. By measuring the oscillation speed or, equivalently, the oscillation phase shift, one can determine how much of the molecule has been captured.

Thus, using the configuration of the sensor with the free-standing membranes makes is possible to increase selectivity of the sensor via adding mechanical stress (mass loading effect) of the molecular or biomolecular layer as an additional parameter of the sensor. In this configuration, the molecular or biomolecular layer (20) is immobilised on the top layer of the PC-HEMT within the open-gate area (17) for the mass loading effect. Using this configuration makes is possible to increase selectivity of the sensor via adding mechanical stress (mass loading effect) of the molecular or biomolecular layer (20) as an additional parameter of the sensor. The very flexible free-standing substrate columns-like membranes can be made in all configurations of the sensor.

Yet further application of the sensors of an embodiment is biomolecular diagnostics including detection of DNA and proteins. In that case, the biomolecular specific layer (20) allows proteins and DNA molecules to be bound or adsorbed and then detected. This biomolecular layer further increases the sensitivity and selectivity of the sensor of an embodiment. The biomolecular layer can be made of various capturing molecules, such as primary, secondary antibodies or fragments thereof against certain proteins to be detected, or their corresponding antigens, enzymes or their substrates, specific DNA sequences complimentary to the DNA to be detected, aptamers, receptor proteins or molecularly imprinted polymers. The biomolecular layer (20) can be immobilised either over the surface of a portion of the recessed 2DEG/2DHG structure or over the entire surface of the PC-HEMT-like area to further improve sensitivity of the sensor for detection of a specific proteins or DNA molecules.

Alternatively, the PC-HEMT of the present invention may not be coated with any molecular layer, such as layer (20), but still be capable of sensing target molecules or biomolecules. Since the sensor of the present invention is clearly capable of overcoming the Debye length limitation, as explained above, sensing of the electric charges with the transistor of the present invention is possible in a contactless manner, when the molecules are at some distance from the surface of the transistor. This allows to overcome a well-known “sensing noise” of any traditional biosensor having reporter molecules attached to the surface of the sensor.

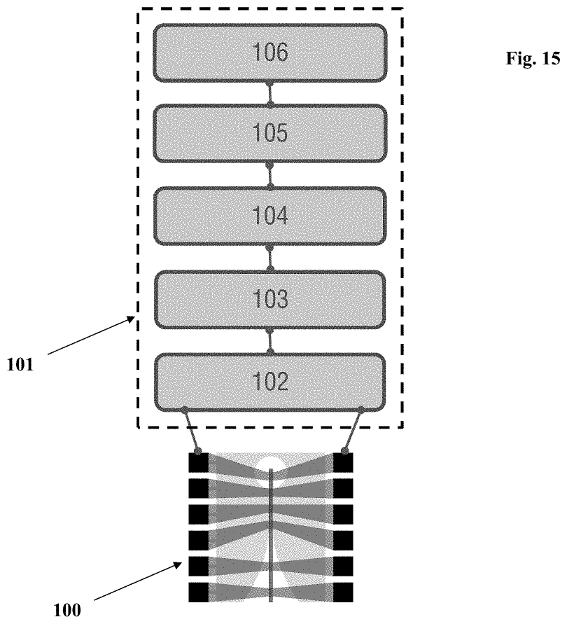

FIG. 15 schematically shows a microelectronic sensor for detection and continuous monitoring of electrical signals in sub-THz and THz frequency ranges, having a remote readout, and comprising the following components (this is a single-transistor solution):

(a) at least one PC-HEMT (100) of the present embodiments with an integrated Vivaldi antenna;
(b) an integrated circuit (101) for storing and processing a signal in a sub-THz or THz frequency domain, and for modulating and demodulating a radio-frequency (RF) signals;
(c) an μ-pulse generator (102) for pulsed RF signal generation;
(d) an integrated DC-RF current amplifier or lock-in amplifier (103) connected to said μ-pulse generator (102) for amplification of the signal obtained from said μ-pulse generator;
(e) an analogue-to-digital converter (ADC) (104) with in-built digital input/output card connected to the amplifier (103) for converting the received analogue signal to a digital signal and outputting said digital signal to a microcontroller unit;
(f) the microcontroller unit (MCU) (105) for processing and converting the received digital signal into data readable in a user interface or external memory;
(g) a wireless connection module (106) for wireless connection of said microelectronic sensor to said user interface or external memory.

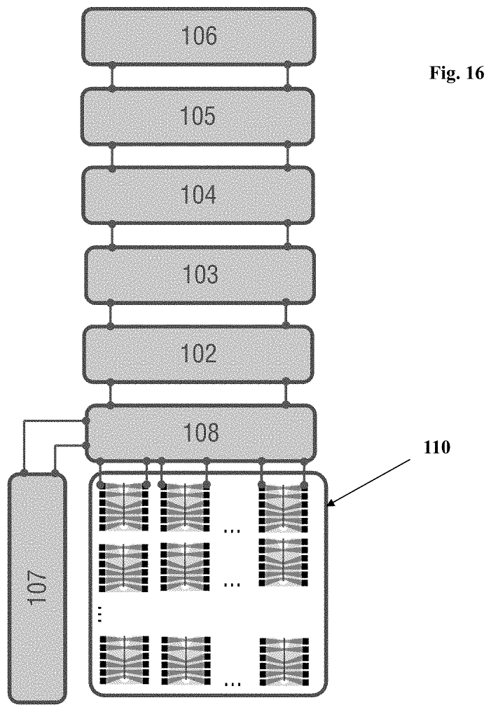

FIG. 16 schematically shows a microelectronic sensor for detection and continuous monitoring of electrical signals in sub-THz and THz frequency ranges, with a remote readout, comprises the following components (this is the DC/RF-based sub-THz and THz antenna transistor-array solution for imaging):

(a) the array of the PC-HEMTs of the present embodiment (110), wherein each PC-HEMT in said array has an integrated Vivaldi antenna and connected to its dedicated electrical contact line;
(b) a row multiplexer (107) connected to said array for addressing a plurality of said transistors (PC-HEMTs) arranged in rows, selecting one of several analogue or digital input signals and forwarding the selected input into a single line;
(c) a column multiplexer (108) connected to said array for addressing a plurality of said transistors (PC-HEMTs) arranged in columns, selecting one of several analogue or digital input signals and forwarding the selected input into a single line;
(d) an integrated circuit for storing and processing said signals in a sub-THz or THz frequency domain, and for modulating and demodulating a radio-frequency (RF) signals;
(e) an μ-pulse generator (102) for pulsed RF signal generation;
(f) an integrated DC-RF current amplifier or lock-in amplifier (103) connected to said μ-pulse generator (102) for amplification of the signal obtained from said μ-pulse generator;
(g) an analogue-to-digital converter (ADC) (104) with in-built digital input/output card connected to the amplifier (103) for converting the received analogue signal to a digital signal and outputting said digital signal to a microcontroller unit;
(h) the microcontroller unit (MCU) (105) for processing and converting the received digital signal into data readable in a user interface or external memory;
(i) a wireless connection module (106) for wireless connection of said microelectronic sensor to said user interface or external memory.

The ADC card (104) may be any suitable analogue-to-digital converter data logger card that can be purchased, for example, from National Instruments® or LabJack®. Optionally, the current amplifier (103) can be operated directly with current flowing via the 2DEG/2DHG channel into the amplifier with small input resistance of 1 MΩ at gain higher than 10⁴ and only 1Ω at gains lower than 200. This setup may directly amplify the electric current modulation in the 2DEG channel originated from external body charges.

In a specific embodiment, the wireless connection module may be a short-range Bluetooth® or NFC providing wireless communication between the sensor and the readout module for up to 20 m. If this connection module is Wi-Fi, the connection can be established with a network for up to 200 nm, while GSM allows the worldwide communication to a cloud. The external memory may be a mobile device (such as a smartphone), desktop computer, server, remote storage, internet storage or cloud.

In some embodiments, the sensors of the present application can be used for portable long-time-operation solution within remote cloud-based service. The portable sensor of an embodiment should have a very small power consumption saving the battery life for a prolong usage. In this case, the non-ohmic high-resistive contacts capacitively connecting the sensor to an electric circuit are preferable. The non-ohmic contacts actually limit an electric current flowing through the 2DEG/2DHG channel by having an electrical resistance 3-4 times higher than the resistance of the 2DEG/2DHG-channel, thereby reducing electrical power consumption without sacrificing sensitivity and functionality of the sensor. Thus, the use of non-ohmic contacts in some embodiments of the sensor of the present application is a hardware solution allowing to minimise the power consumption of the device. In another embodiment, the power consumption of the device can be minimised using a software algorithm managing the necessary recording time of the sensor and a battery saver mode, which limits the background data and switches the wireless connection only when it is needed.

In some embodiments, a method for chemical sensing and biomolecular diagnostics comprises the following steps:

(1) Subjecting a sample to be tested to the microelectronic senor of the present embodiments;
(2) Recording electrical signals received from the sample with the microelectronic sensor in a form of a source-drain electric current of the microelectronic sensor over time (I_{DS} dynamics) and/or measuring S11-S12 parameters of the microelectronic sensor over time (S11-S12 dynamics);
(3) Transmitting the recorded signals from said microelectronic sensor to an external memory for further processing; and
(4) Converting the transmitted signals to digital signals and processing the digital signals in the external memory, comparing said I_{DS} dynamics and/or S11-S12 dynamics with negative control chemical or biomolecular I_{DS} or S11-S12-transfer waveforms stored in the external memory, and extracting chemical or biomolecular information from said waveforms in a form of readable data, thereby detecting and/or identifying a particular chemical or biological compound (target, analyte) in the sample and measuring its concentration.

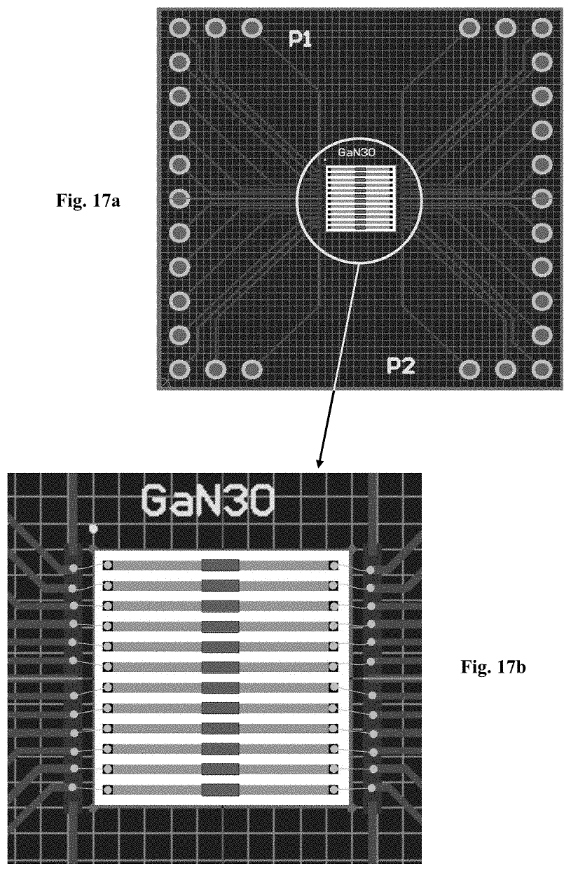

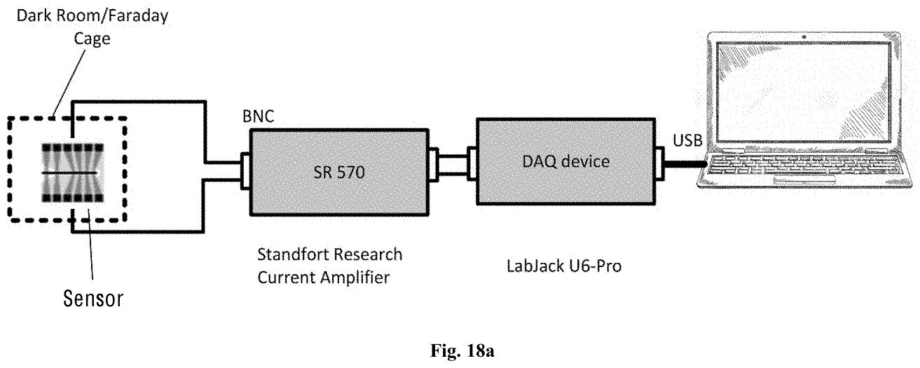

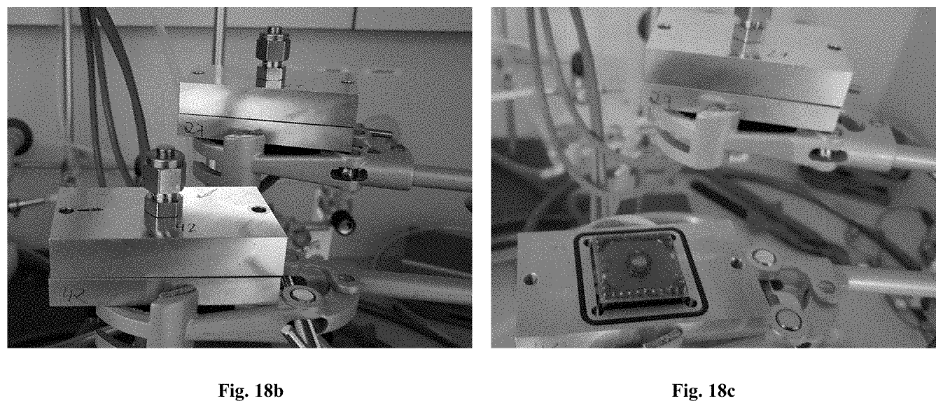

Reference is now made to FIGS. 17a-17b showing the exemplary sensor layout of the present invention. The exemplary sensor has 2×12 contact pads. The experimental sensor setup is schematically shown in FIG. 18a, while FIGS. 18b and 18c show the photographs of this setup in the lab. In the following experiments, all the reagents, analytes, buffers, kits and bioassays were kindly provided by EUROIMMUN AG (Germany). All assay kits used in the experiments below contained washing buffer, capture analyte solution, blocking solution and target analyte solution. The working experimental procedure was as follows:

1) Rinsing after encapsulation was only done if the sensor surface was extremely dirty. For this purpose, deionised water was used to carefully rinse the chip. If the surface was looking good, no surface cleaning was done.
Positive control: Coat the sensor surface with the capture molecule solution. Concentrations and coating procedures were provided by EUROIMMUN AG. The immobilisation was performed for either two hours at room temperature or overnight at 4° C. in the fridge.
Negative control: Incubate the sensor with a buffer (same buffer, in which the capture analyte was diluted to its final concentration) for either two hours at room temperature or overnight at 4° C. in a fridge.
2) Wash the positive and negative sensors carefully with an assay buffer after the immobilisation is completed.
3) Block the positive and negative sensors with a blocking buffer for one hour at room temperature.
4) Wash the positive and negative sensors carefully with the assay buffer afterwards.
5) Pipette 40 μL of the assay buffer onto the sensor surface and start measuring both sensors (positive and negative control) in parallel.
6) After 10 min, add 40 μL of a target analyte solution (concentration of the target analyte was given by EUROIMMUN AG) and measure the resulted solution with the sensor*.
*Note: After adding the target analyte, the sensor is not washed anymore.
7) Stop the measurements when the signal gets stable.

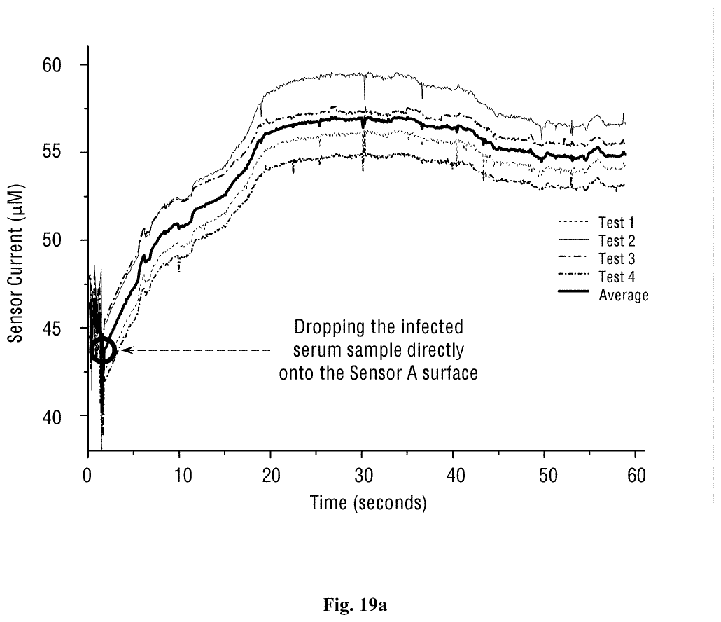

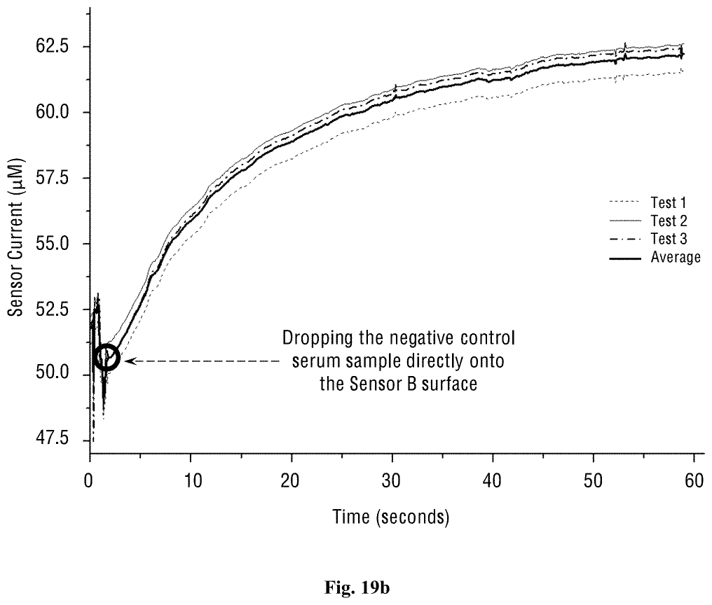

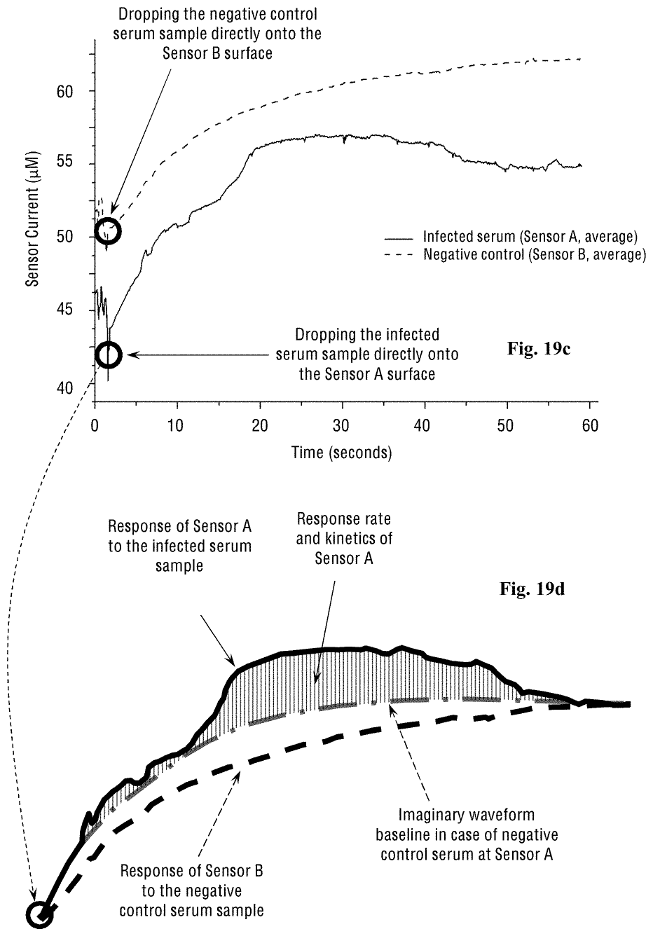

In the first experiment demonstrated in the present disclosure, Sensors A and B are the same sensors of the present invention having the same configuration. An antigen of Lime borreliosis was initially applied to the sensor surface. Sensor A received a drop of human blood serum having a very small amount of Lime borreliosis, containing the corresponding antibody with four different concentrations in the concentration range up to 100 pg/ml. Sensor B was used as a control receiving a drop of clean human serum without the infection. FIG. 19a shows the results of this experiment (increase of the senor current) upon introducing a drop of the infected blood serum onto the surface of the Sensor A. The four curves shown in the figure represent the four serum samples having different concentrations of Lime borreliosis in the concentration range up to 100 pg/ml, while the fifth (bold) curve shows the average of these four tests. FIG. 19b shows the reference results upon introducing a drop of the negative control serum onto the Sensor B surface. The three curves shown in the figure represent three different samples of a clean human serum, while the fourth (bold) curve shows the average of these three tests. FIG. 19c shows the comparison of the average curves for Sensors A and B in the first experiment, while FIG. 19d shows the normalised comparison of Sensors A and B in the first experiment, with graphical calculation of response rate and kinetics of Sensor A toward the infection.

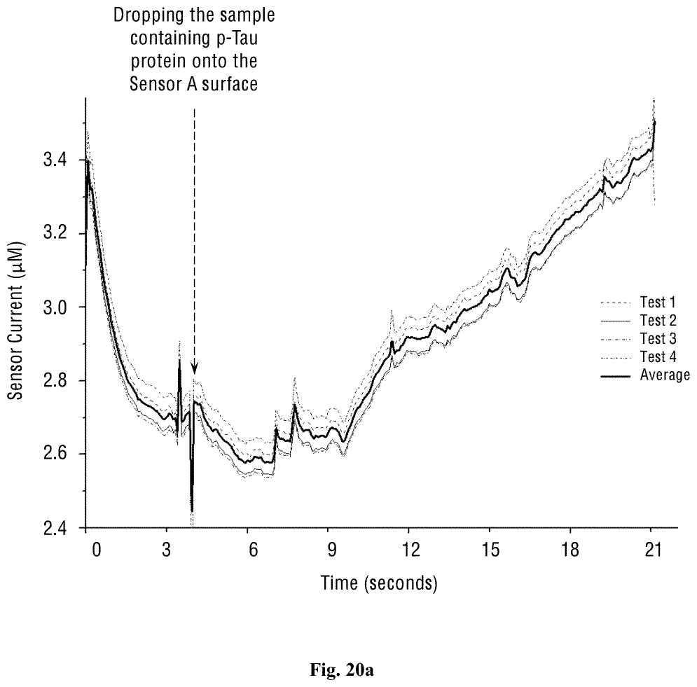

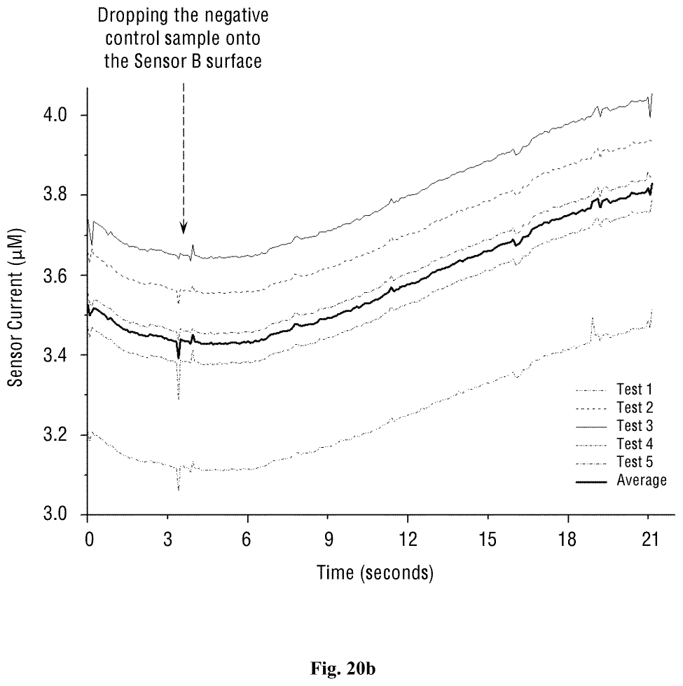

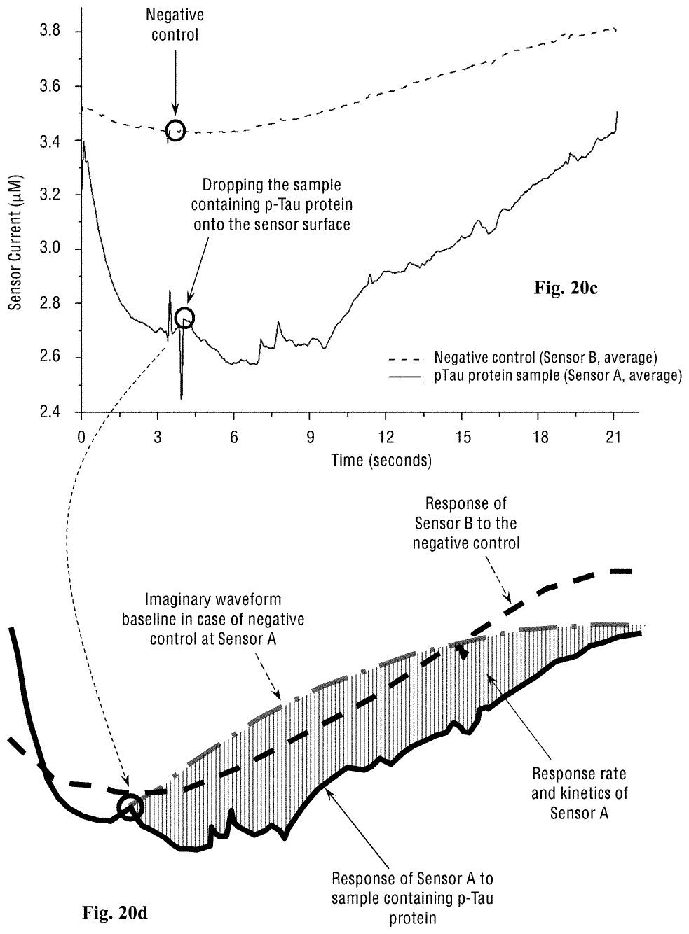

The second experiment demonstrated the similar effect with the p-Tau protein, levels of which are used in the prediction of Alzheimer's disease. Sensor A pre-treated with the anti-Tau antibody was exposed to positive p-Tau protein serum samples with four different concentrations in the concentration range up to 100 pg/ml. Sensor B in this case was used as a negative control receiving a drop of human blood serum with almost no p-Tau protein (the concentration of the p-Tau protein was 0.05 pg/ml in the control sample). FIGS. 20a-20d show the results of this experiment.

FIG. 20a shows the I_{DS} dynamics of the Sensors A and B in the second experiment with the p-Tau protein. Sensor A was exposed to positive p-Tau protein serum samples with four different concentrations in the concentration range up to 100 pg/ml, while the fifth (bold) curve shows the average of these four tests. FIG. 20b shows the results of the second experiment with Sensor B (its I_{DS} dynamics) that was used as a negative control receiving a drop of human blood serum with the concentration of the p-Tau protein 0.05 pg/ml in the control sample. The five curves shown in the figure represent five different samples of a clean serum, while the sixth (bold) curve shows the average of these five tests. FIG. 20c shows the comparison of the average curves for Sensors A and B in the second experiment. FIG. 20d shows the normalised comparison of Sensors A and B in the second experiment, with graphical calculation of response rate and kinetics of Sensor A toward the presence of the protein.

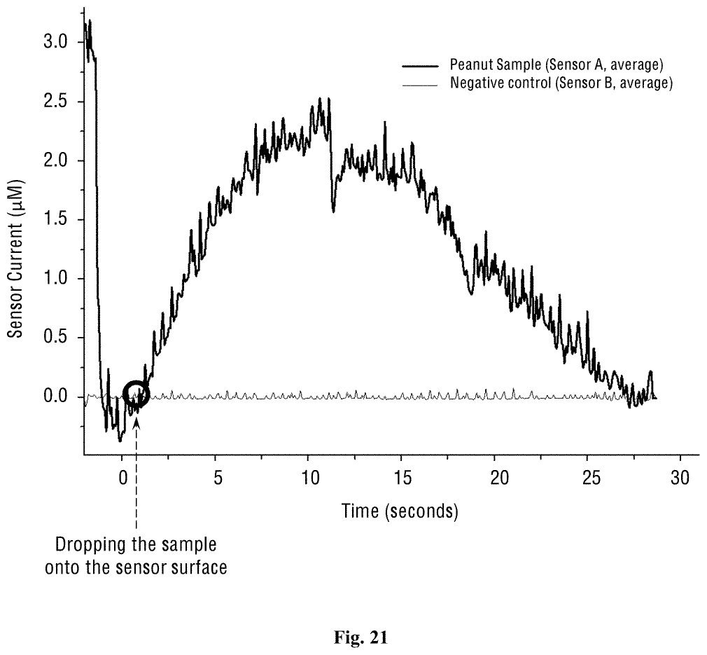

FIG. 21 shows the results of the third experiment with Sensors A and B in the form of the measured I_{DS} dynamics. A peanut sample was introduced onto the surface of Sensor A for allergen detection, where Sensor B was used as a negative control. Each curve shown in the figure is an average of three independent tests. The curves were normalised to baseline for comparison.

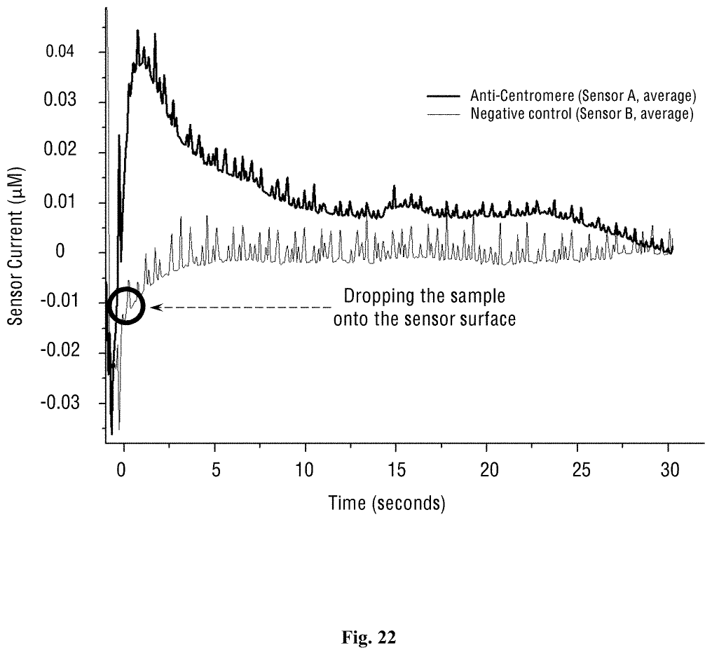

FIG. 22 shows the results of the fourth experiment with Sensors A and B in the form of the I_{DS} dynamics. A sample containing anti-centromere antibodies, which are autoantibodies specific to centromere and kinetochore function, was dropped on the surface of Sensor A for their detection, where Sensor B was used as a negative control. Each curve shown in the figure is an average of two independent tests. The curves were normalised to baseline for comparison.

As noted above, the main limitation in the DC readout mode is the Debye screening of charges. Moreover, the DC readout is not suitable for various functionalised surfaces and mainly depends on the charge carried by the target molecule. The present inventors proposed to overcome these limitations by adding the AC readout with a frequency sweep up to 1 MHz or higher. Opposite to the DC readout, the charges of the target molecules have a negligible influence on the sensing in the AC mode. In addition, the AC readout can detect the presence of the bound molecules. The AC sensing mode has the same basis as the impedance spectroscopy. It shows the change of the sensor's surface capacitance and resistance which contains information about the binding of the target molecule, as well as the ‘number’ (concentration) of the bound molecules.

Thus, the AC electronic readout combined with the DC readout is useful for enzymatic, electrochemical and affinity sensing when both charged and uncharged molecules are involved. When operated at higher frequencies (more than 1 MHz), the problematic Debye screening can be overcome, and also larger molecules can be sensed.

In a further aspect of the present invention, the combined transducer principle defined herein as a “triple readout” includes: DC electronic readout of the sensor, AC electronic readout of the sensor and temperature sensing. The PC-HEMT-based sensor of the present invention therefore further comprises a reference electrode and characterised with respect to its electronic properties and to the measurement configuration for molecular sensing applications. The main features of the sensors of the present invention are determined by the transfer characteristics and the output characteristics at room temperature. The transfer characteristics shows the drain current of the PC-HEMTs as a function of their source voltage at constant drain-source voltages.

In general, the term “transfer function” (TF) is a mathematical representation to describe inputs and outputs of black box models. In order to describe the frequency response of the sensor, a counter electrode and the first amplifier stage are considered as a black box element with a certain frequency response. Since the analogue transistor amplification is exploited in the present invention, the instant model is described with a term “transistor transfer function” (TTF). The TTF is defined as a mathematical ratio between the input (Vₛₜᵢₘ) and the output signal (Vₒᵤₜ) of an electrical, frequency-dependent system. Its frequency response H(jω) is defined as follows:

$$H ( j ω ) = \frac{{V}_{out} ( j ω )}{{V}_{stim} ( j ω )} ,$$

wherein ω is the angular frequency and j is the imaginary unit.

The TTF can be used to investigate impedance (defined as the ratio between voltage and applied current) or capacitance (defined as the capability of a capacitor to store charges) changes, caused by binding of molecules onto the PC-HEMT surface. This detection of analytes was reported in several publications even though the theory, on which the TTF relies, is still under discussion, because for each particular device and amplifier design, there are many parasitic side parameters that have an extremely drastic effect on the TTF. A universal model is therefore difficult to establish. However, the present inventors have demonstrated that it is possible, to investigate, for instance the DNA hybridization, protein binding and to perform cell recordings by measuring this function.

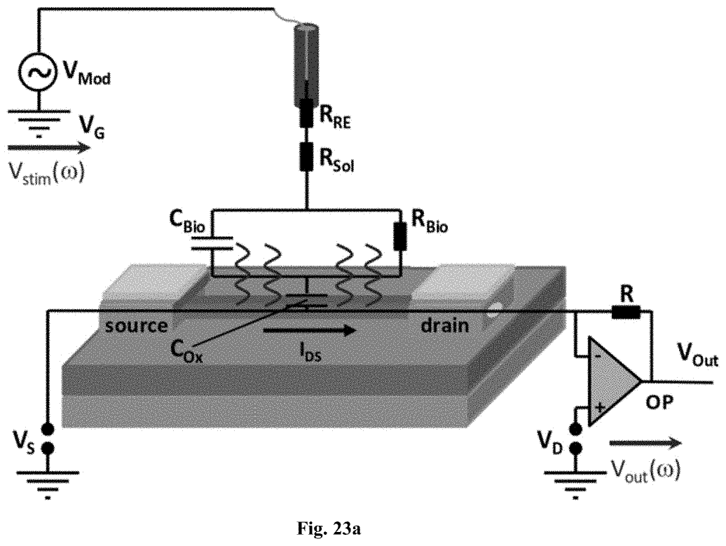

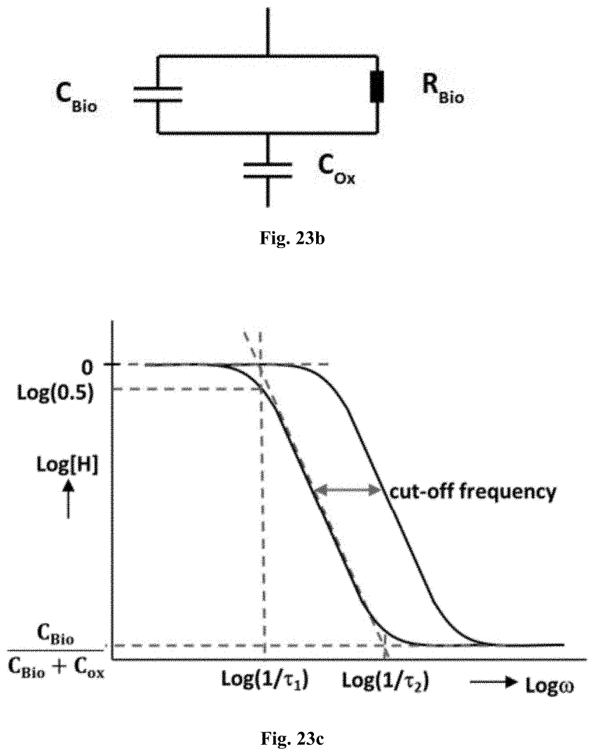

In general, binding of molecules onto the surface of the PC-HEMT top layer leads to a capacitance change and consequently, to an impedance change of the solid-liquid interface of the transistor for the reasons explained above. For better understanding of the TTF of the PC-HEMT-based sensor of the invention, its simplified equivalent circuit is shown in FIG. 23a. This is a very crude approximation excluding all parasitic parameters of the reference electrode, electrolyte conductivity, transistor's feed lines and amplifier characteristics. In the most simplistic approach, the transistor's impedance is represented by the capacitance C_{Bio} and the resistance R_{Bio}, which are in parallel to each other and in series with the capacitance. For a more complete modelling, the capacitance of the common source contact leads in parallel to the capacitance of the drain contact lead needs to be included. By binding of biomolecules to the PC-HEMT surface in the open gate area (17), only C_{Bio} and the resistance R_{Bio} are affected. Therefore, to describe the main response of the system, only the simplified circuit as shown in FIG. 23b is discussed herein.

As shown in FIG. 23a, the molecular/biomolecular layer (20) is immobilised on the PC-HEMT surface. The molecules or biomolecules inside this layer (20) can be described as capacitance C_{Bio} and resistance R_{Bio}. Due to binding of complementary target molecules to the receptor molecules in the layer (20), the impedance of the system and, hence, the TTF are changed. The theoretical TTF corresponding to the circuit shown in FIG. 23b is displayed on FIG. 23c, wherein the left curve is for a bare transistor surface (without the molecular/biomolecular layer) and the right curve is for the transistor surface with the molecular/biomolecular layer (20). Two time constants τ₁ and τ₂ can be evaluated from the theoretical transfer function H(jω):

τ₁=R_{Bio}(C_{Bio}+C_{Ox})=τ₂+R_{Bio}C_{Ox}

τ₂=R_{BiO}C_{Bio}

wherein τ₂ actually represents the relaxation time of the biomolecular layer.

The nature of the impedance change of the biomolecular layer is having many components. The size, isoelectric point and hence, the pH value of the test solution, the charge, distance, orientation, and packing density of the molecules are influencing this. In addition, the shape of the measured TTF curve depends on other (non-tested) parameters such as the reference electrode resistance R_{RE}, solution resistance R_{Sol} and capacitances of the contact leads. The shift between these two curves is the so-called “cut-off frequency” or “band pass behaviour”. From this frequency shift, the concentration of the molecules can be calibrated.

It is well known, that the shift of the TTF and, therefore, the size of the cut-off frequency are more pronounced in much lower concentrated electrolyte solutions. Therefore, the Debye screening of charges is also one component in the TTF approach, but not a dominating component like in the DC recording alone with the sensor of the invention.

The DC electronic readout is based on the transfer characteristics and is carried out in a liquid medium. The sensor in a DC readout mode is biased by a certain drain-source voltage while a voltage sweep is done through a reference electrode, and senses the charges at the sensor surface functionalised with the molecular/biomolecular layer (20). The resulting transfer characteristics reflects the characteristic behaviour of the sensor, as well as its surface condition, and is used to detect target molecules on the functionalised sensor surface. When target molecules bind to the sensor surface, electric current changes of the sensor become dependent on the charge of the target molecules at the surface and consequently on their concentration. Negatively charged molecules leads to a shift of the transfer characteristics to the right and positively charged molecules cause a shift to the left.

In conclusion, what makes the sensor of the present embodiments particularly useful and unique is the combination of the PC-HEMT and Vivaldi antenna in one single transistor. Numerical simulations in the sub-THz and THz frequency range of 30 GHz to 300 THz were conducted and the initial sets of measurements were made. The Vivaldi antenna of the sensor chip of the embodiments is passively receiving the sub-THz or THz signals from the liquid, solid or gas medium in approximately 30 GHz to 300 THz range.

By applying additional plasmonic filter structures of any kind, the frequency may be precisely adjusted to a frequency of choice. The detection principle is based on the field effect current modulation in a DC-mode within the 2DEG conducting channel of the PC-HEMT achieved by Vivaldi-shaped gate antenna. Once a sample to be tested is applied to the sensor, the Vivaldi-shaped gate antenna instantly and strongly concentrates the electric field strength of the sub-THz and THz radiation in a very small area above the ultra-charge sensitive 2DEG channel, thereby affecting its conductivity in the DC-mode by accumulation effect.

In addition to the DC mode, the AC electronic readout can be used. Their combination is useful for enzymatic, electrochemical and affinity sensing when both charged and uncharged molecules are involved. When operated at higher frequencies (more than 1 MHz), the problematic Debye screening can be overcome, and also larger molecules can be sensed.

1. A method for chemical sensing and biomolecular diagnostics comprising:
(1) Applying a sample to be tested to a microelectronic senor;
(2) Recording electrical signals received from the sample with the microelectronic sensor in a form of a source-drain electric current of the microelectronic sensor over time (I_{DS} dynamics);
(3) Transmitting the recorded signals from said microelectronic sensor to an external memory for further processing; and
(4) Converting the transmitted signals to digital signals and processing the digital signals in the external memory, comparing said I_{DS} dynamics with negative control chemical or biomolecular I_{DS} waveforms stored in the external memory, and extracting chemical or biomolecular information from said waveforms in a form of readable data, thereby detecting and/or identifying a particular chemical or biological compound (target, analyte) in the sample and measuring its concentration;
characterised in that said microelectronic sensor comprises at least one open-gate pseudo-conductive high-electron mobility transistor for amplifying signals in the frequency range of 30 GHz to 300 THz, said transistor comprising:
(i) a multilayer heterojunction structure being composed of III-V single-crystalline or poly-crystalline semiconductor materials and deposited on a substrate layer or placed on free-standing membranes, said structure comprising at least one buffer layer and at least one barrier layer, said layers being stacked alternately;
(ii) a conducting channel comprising a two-dimensional electron gas (2DEG) or a two-dimensional hole gas (2DHG), formed at the interface between said buffer layer and said barrier layer, and upon applying a bias to said transistor, becoming capable of providing electron or hole current, respectively, in said transistor between source and drain contacts;
(iii) the source and drain contacts connected to said 2DEG or 2DHG conducting channel and to electrical metallisations for connecting said transistor to an electric circuit; and
(iv) a Vivaldi antenna electrode placed on the top layer between said source and drain contact areas in an open gate area of the transistor and capable of detecting electrical signals in the frequency range of 30 GHz to 300 THz
said transistor is characterised in that the thickness (d) of the top layer of said heterojunction structure in the open gate area is 5-9 nanometres (nm) which corresponds to the pseudo-conducting current range between normally-on and normally-off operation mode of the transistor, and the surface of said top layer has a roughness of about 0.2 nm or less, wherein the combination of said thickness and said roughness of the top layer allows to observe the pseudo-conducting current in said transistor.

2. The method of claim 1, wherein said transistor further comprising at least one molecular or biomolecular layer immobilised within the open gate area of said transistor and capable of binding or adsorbing target (analyte) gases, chemical compounds or biomolecules from the environment.

3. The method of claim 1, wherein said transistor is not coated with a molecular or biomolecular layer and is capable of remotely detecting target (analyte) gases, chemical compounds or biomolecules from the environment.

4. The method of claim 1, wherein said source and drain contacts of said transistor are ohmic.

5. The method of claim 1, wherein said electrical metallisations of said transistor are capacitively-coupled to said 2DEG or 2DHG conducting channel for inducing displacement currents, thus resulting in said source and drain contacts being non-ohmic.

6. The method of claim 1, wherein said transistor further comprising a dielectric layer deposited on top of said multilayer hetero junction structure.

7. The method of claim 1, wherein said III-V single-crystalline or polycrystalline semiconductor materials are GaN/AlGaN, and said multilayer heterojunction structure comprising either:
(a) (i) one top AlGaN layer recessed in an open gate area of the transistor to the thickness of 5-9 nm and having the surface roughness of 0.2 nm or less, and (ii) one bottom GaN buffer layer; said layers have Ga-face polarity, thus forming the two-dimensional electron gas (2DEG) conducting channel in said GaN layer, close to the interface with said AlGaN layer; or
(b) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer; or
(c) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
(d) (i) one top AlGaN layer recessed in an open gate area of the transistor to the thickness of 5-9 nm and having the surface roughness of 0.2 nm or less, and (ii) one bottom GaN buffer layer; said layers have N-face polarity, thus forming a two-dimensional hole gas (2DHG) conducting channel in the GaN buffer layer, close to the interface with said AlGaN barrier layer.

8. The method of claim 1, wherein the thickness of the top layer recessed in the open gate area of said transistor is 6-7 nm.

9. The method of claim 8, wherein the thickness of the top layer recessed in the open gate area of said transistor is 6.2 nm to 6.4 nm.

10. The method of claim 1, wherein the surface roughness of the top layer recessed in the open gate area of said transistor is 0.1 nm or less.

11. The method of claim 10, wherein the surface roughness of the top layer recessed in the open gate area of said transistor is 0.05 nm or less.

12. The method of claim 1, wherein said chemical is a toxic metal, such as chromium, cadmium or lead, a regulated ozone-depleting chlorinated hydrocarbon, a food toxin, such as aflatoxin, or shellfish poisoning toxin, such as saxitoxin or microcystin, a neurotoxic compound, such as methanol, manganese glutamate, nitrix oxide, Botox, tetanus toxin or tetrodotoxin, oxybenzone, Bisphenol A, or butylated hydroxyanisole, an explosive, such as picrate, nitrate, trinitro derivative, such as 2,4,6-trinitrotoluene (TNT), 1,3,5-trinitro-1,3,5-triazinane (RDX), trinitroglycerine, N-methyl-N-(2,4,6-trinitrophenyl)nitramide (nitramine or tetryl), pentaerythritol tetranitrate (PETN), nitric ester, azide, derivate of chloric and perchloric acids, fulminate, acetylide, and nitrogen rich compound, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosive, such as a decomposition gas or taggant.

13. The method of claim 1, wherein said biological compound is a biological pathogen, such as a respiratory viral or bacterial pathogen, an airborne pathogen, a plant pathogen, a pathogen from infected animals or a human viral pathogen.

14. The method of claim 2, wherein said molecular or biomolecular layer of said transistor is a cyclodextrin, 2,2,3,3-tetrafluoropropyloxy-substituted phthalocyanine or their derivatives, or said molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

15. The method of claim 1, wherein said microelectronic sensor is suitable for detection and continuous monitoring of electrical signals in the frequency range of 30 GHz to 300 THz and consequently, for chemical sensing and biomolecular diagnostics in said frequency range, said sensor having a remote readout and comprising:
(a) at least one said transistor (100);
(b) an integrated circuit (101) for storing and processing a signal in a sub-THz or THz frequency domain, and for modulating and demodulating a radio-frequency (RF) signals;
(c) an μ-pulse generator (102) for pulsed RF signal generation;
(d) an integrated DC-RF current amplifier or lock-in amplifier (103) connected to said μ-pulse generator (102) for amplification of the signal obtained from said μ-pulse generator;
(e) an analogue-to-digital converter (ADC) (104) with in-built digital input/output card connected to the amplifier (103) for converting the received analogue signal to a digital signal and outputting said digital signal to a microcontroller unit;
(f) the microcontroller unit (MCU) (105) for processing and converting the received digital signal into data readable in a user interface or external memory; and
(g) a wireless connection module (106) for wireless connection of said microelectronic sensor to said user interface or external memory.

16. The method of claim 1, wherein said microelectronic sensor is suitable for detection and continuous monitoring of electrical signals in the frequency range of 30 GHz to 300 THz and consequently, for chemical sensing and biomolecular diagnostics in said frequency range, said sensor having a remote readout and comprising:
(a) an array (110) of said transistors (100), wherein each transistor (100) in said array (110) has an integrated Vivaldi antenna and connected to its dedicated electrical contact line;
(b) a row multiplexer (107) connected to said array (110) for addressing a plurality of said transistors (100) arranged in rows, selecting one of several analogue or digital input signals and forwarding the selected input into a single line;
(c) a column multiplexer (108) connected to said array (110) for addressing a plurality of said transistors (100) arranged in columns, selecting one of several analogue or digital input signals and forwarding the selected input into a single line;
(d) an integrated circuit for storing and processing said signals in a sub-THz or THz frequency domain, and for modulating and demodulating a radio-frequency (RF) signals;
(e) an μ-pulse generator (102) for pulsed RF signal generation;
(f) an integrated DC-RF current amplifier or lock-in amplifier (103) connected to said μ-pulse generator (102) for amplification of the signal obtained from said μ-pulse generator;
(g) an analogue-to-digital converter (ADC) (104) with in-built digital input/output card connected to the amplifier (103) for converting the received analogue signal to a digital signal and outputting said digital signal to a microcontroller unit;
(h) the microcontroller unit (MCU) (105) for processing and converting the received digital signal into data readable in a user interface or external memory; and
(i) a wireless connection module (106) for wireless connection of said microelectronic sensor to said user interface or external memory.
